(19) United States
(12) Patent Application Publication
(10) Pub. No.: US 2025/0195687 A1
(43) Pub. Date: Jun. 19, 2025

(54) PEPTIDE-BASED PHOSPHATE BINDER FOR THE TREATMENT OF HYPERPHOSPHATEMIA

(71) Applicant: B. G. NEGEV TECHNOLOGIES AND APPLICATIONS LTD., Beer-Sheva (IL)

(72) Inventors: Moshe GOTTLIEB, Omer (IL); Roza TRUBERMAN, Holon (IL); Michal ZAIDEN, Kibbutz Shoval (IL)

(21) Appl. No.: 18/846,323

(22) PCT Filed: Mar. 19, 2023
(86) PCT No.: PCT/IL2023/050282

§ 371 Date: Sep. 12, 2024

Related U.S. Application Data
(60) Provisional application No. 63/322,462, filed on Mar. 22, 2022.

(51) Int. Cl. A61K 47/69; A61K 47/62

(52) U.S. Cl. CPC A61K 47/6929; A61K 47/62; A61K 47/6923

(57) ABSTRACT

Provided herein are silica nanoparticles conjugated to a phosphate binding hexapeptide (PBH) via a linker amino acid residue and methods of making the same. Further provided are compositions including the nanoparticles and uses thereof in the treatment of hyperphosphatemia.

### FIELD OF THE INVENTION

The present invention relates to silica-particles conjugated to specific phosphate binding peptides, compositions comprising the same and uses thereof in the treatment of hyperphosphatemia.

### BACKGROUND OF THE INVENTION

Phosphorus is present in nearly all foods, and the gastrointestinal (GI) absorption of dietary forms of phosphate is very efficient. With low dietary intake, 80-90% of phosphate is absorbed. When intake is greater than 10 mg phosphate/kg body weight/day, 70% is absorbed. Average daily dietary intake varies from 800-1500 mg. Absorption occurs mainly in the jejunum, although some absorption occurs throughout the entire GI tract. Phosphate homeostasis is a highly regulated process. The movement of phosphate in and out of bone, the reservoir containing most of the total body phosphate, is generally balanced. Renal excretion of excess dietary phosphate intake ensures maintenance of phosphate homeostasis. Phosphate is a predominantly intracellular anion. Most intracellular phosphate is either complexed or bound to proteins or lipids. In response to kinases and phosphatases, these phosphate ions attach and detach from different molecules, forming a constantly shifting pool.

Changes in serum phosphate levels are generally proportional to dietary intake. To keep serum phosphate levels in balance, several tissues sense changes in phosphate levels and produce endocrine factors such as parathyroid hormone (PTH) and fibroblast growth factor-23 (FGF23). PTH is activated by binding to the PTH receptor and FGF23 requires both the FGF receptor and the co-factor Klotho. PTH and FGF23 act on the kidneys to decrease phosphate reabsorption.

These processes also cause a decrease in vitamin D produced in the kidney and are presumed to reduce phosphate absorption from the intestines and generate a negative feedback loop to reduce PTH and FGF23. The result is increased phosphate excretion in the urine and decreased absorption in the GI tract, which brings serum Pi levels back into balance.

Hyperphosphatemia is a disorder characterized by abnormally high serum phosphate levels. It can result from increased phosphate intake, decreased phosphate excretion, or conditions that shifts intracellular phosphate to extracellular space. Hyperphosphatemia is a major concern because of its association with cardiovascular morbidity and mortality among chronic kidney disease (CKD) and dialysis patients. Furthermore, increased levels of serum phosphate have been associated with rewired cell signaling pathways, impaired bone mineralization, infertility, arteriosclerosis, and accelerated aging. Elevated phosphorus levels have been associated with premature aging, cell apoptosis, cardiac remodeling and dysfunction, tumorigenesis, and arthrosclerosis.

Currently used treatments for hyperphosphatemia include dietary awareness and control; Dialysis; and various types of phosphate binders (PhB). The majority of clinically used PhB are metal ion salts that combine with the phosphate in the GI tract and decrease the free phosphate, which is eliminated in the feces, thus limiting intestinal absorption. However, the dissolved ions tend to be absorbed through the intestine walls, causing severe health risks and problems. Aluminum containing PhB are effective at binding phosphate. However, aluminum concentration in dialysate water correlated with the incidence of aluminum bone disease. Calcium based PhB are widely used, however, they increase the risk of hypercalcemia and progression of vascular calcification. Lanthanum carbonate is a non-calcium-based phosphate binder, which contains lanthanum posing a concern regarding its accumulation in the liver. Sevelamer hydrochloride is a non-absorbable polymeric phosphate binder effective in reducing phosphorus, however, it exhibits various gastrointestinal side-effects, such as constipation, lower gastrointestinal bleeding, and metabolic acidosis. Peptide based phosphate binders have also been proposed as potential treatment, however, such peptides would not necessarily function under physiologically relevant pH (Bianchi, et. al. (2012), A Synthetic Hexapeptide Designed to Resemble a Proteinaceous P-Loop Nest Is Shown to Bind Inorganic Phosphate. Proteins Struct. Funct. Bioinforma. 2012, 80 (5), 1418-1424); Fowler, et. al. (2021), Harnessing Peptide Binding to Capture and Reclaim Phosphate. J. Am. Chem. Soc. 2021, 143 (11), 4440-4450).

Thus, due to the shortcomings in existing phosphate binding (PhB) remedies there is a need to develop a non-systemic PhB active in the physiological gastric and intestine pH. Accordingly, there is a need in the art for improved compositions for treating hyperphosphatemia, which are particularly active in the physiological gastric and intestine pH and are thus efficient, exhibit reduced side effects and are cost effective.

### SUMMARY OF THE INVENTION

According to some embodiments, there are provided advantageous silica particles, conjugated to specific phosphate binding peptides, in particular, via a cysteine amino acid, wherein the conjugated particles are particularly active in physiologically relevant pH ranges. In some embodiments, the particles are nano-particles and the peptide is a hexapeptide having an additional cysteine residue at its N-terminus, allowing covalent binding to the surface of the silica nano-particles. According to some embodiments, the advantageous conjugated silica particles and composition including them can be efficiently used for the treatment of hyperphosphatemia.

According to some embodiments, provided herein are novel phosphate binding (PhB) particles based on biocompatible silica nanoparticles (NP) decorated with tethered phosphate-binding peptides (NP-pep), which exhibit reduced side effects and increased hyperphosphatemia treatment efficiency, as compared to other hyperphosphatemia treatments. The advantageous NP-pep particles disclosed herein are non-systemic due to their size and are thus highly efficient while exhibiting reduced side effects.

According to some embodiments, further provided herein a method for preparing of the advantageous NP-pep disclosed herein. In some embodiments, the preparation method/process includes the attachment/linking of the phosphate-binding peptide to the silica surface, which is carried out by a three steps synthesis. The first step involves the replacement of the hydroxyl groups on the silica surface with amine groups. In the second step, maleic anhydride is reacted with the amine groups to yield maleimide end group. Finally, a hexapeptide with a cysteine amino acid end group is attached to the maleimide functionalized NP. As exemplified herein, accurate quantification of the active groups attached to the NPs following each synthesis step performed, to evaluate the efficiency of the process.

In some embodiments, as exemplified herein, in order to evaluate the efficiency of phosphate-binding in the GI track, tests were performed in gastric and intestinal fluid simulants at fluid pH and compositions matching the different physiological conditions, to further substantiate the activity and efficiency of the NP-pep.

According to some embodiments, as exemplified herein, the synthesis of the SiO2-peptide was thoroughly investigated using several analytical methods, including elemental analysis, XPS, TGA, and 1H qNMR, with TGA and 1H qNMR emerged as the most reliable methods for surface characterization, and coverage quantification.

According to some embodiments, as exemplified herein, a specific methodology was developed to allow accurate determination of particle coverage.

According to some embodiments, advantageously, the CysHex peptides are attached to the silica NP surface at a surface concentration of about 0.5 peptide molecules per nm² of silica surface area.

According to some embodiments, as exemplified herein, advantageously, phosphate binding to the SiO2-CysHex PhB evaluated by means of a colorimetric technique, exhibited the highest phosphate binding at pH values around 6, which is the physiologically relevant range of values. Further, advantageously, the amount of phosphate captured by the particles varied between 1.3 to 2.6 mg phosphate/gr PhB under different conditions.

According to some embodiments, as further exemplified herein, in order to evaluate the pharmacological properties of the novel PhB, its binding ability was compared to that of a clinically used agent (lanthanum carbonate), which exhibited the same order of magnitude of phosphate binding efficiency of the SiO₂-CysHex particles is in the as lanthanum carbonate and being twice as effective in the fed state.

According to some embodiments, there is provided a composition including silica nanoparticles conjugated to a phosphate binding hexapeptide (PBH) via a linker amino acid residue (SiO2-CysHex NP).

According to some embodiments, the linker amino acid residue includes a Cysteine amino acid. In some embodiments, the hexapeptide have an amino acid sequence as denoted by SEQ ID NO: 1. In some embodiments, the hexapeptide included a Cys linker amino acid residue at the N-terminal thereof, wherein the conjugated peptide has an amino acid sequence as denoted by SEQ ID NO: 2.

According to some embodiments, the silica nanoparticles may have an average size of about 5-200 nm. According to some embodiments, the silica nanoparticles may have an average size of about 7-15 nm.

According to some embodiments, the silica nanoparticles may have an average surface area in the range of about 100-400 m²/gr. According to some embodiments, the silica nanoparticles may have an average surface area in the range of about 200 m²/gr.

According to some embodiments, each nanoparticle may be conjugated to at least about 200 PBH molecules.

According to some embodiments, the composition may be for use in treating Hyperphosphatemia.

According to some embodiments, the composition may be formulated for systemic administration. In some embodiments, the administration is enteral. In some embodiments, the administration is oral administration.

According to some embodiments, the composition may be for use in treating Hyperphosphatemia of a chronic kidney disease (CKD) patient.

According to some embodiments, there is provided a method of treating Hyperphosphatemia, the method includes administering a pharmaceutically effective amount of the composition of SiO2-CysHex NPs to a subject in need thereof. According to some embodiments, the subject is a subject afflicted with chronic kidney disease (CKD),

According to some embodiments, there is provided a process for the preparation of silica nanoparticles conjugated to a phosphate binding hexapeptide (PBH) (SiO₂-CysHex NPs), the process includes the steps of:
i) substituting hydroxyl group(s) on the silica nanoparticles surface with amine group(s);
ii) reacting maleic anhydride with the amine group(s) of the silica nanoparticles surface to yield maleimide end group(s); and
iii) attaching an hexapeptide having a cysteine amino acid end group linker to the maleimide functionalized nanoparticles, via said cysteine amino acid end group linker, to thereby form Silica-CysHex particles.

In some embodiments of the process, the hexapeptide may include an amino acid sequence as denoted by SEQ ID NO: 1.

In some embodiments of the process, the silica nanoparticles have an average size of about 5-200 nm. In some embodiments, the silica nanoparticles may have an average size of about 7-15 nm.

In some embodiments of the process, the silica nanoparticles may have an average surface area in the range of about 100-400 m²/gr. In some embodiments of the process, the silica nanoparticles may have an average surface area of about 200 m²/gr.

In some embodiments of the process, each nanoparticle may be conjugated to at least about 200 hexapeptide molecules.

In some embodiments, the process may further include a step of drying the resulting Silica-CysHex particles (SiO₂-CysHex NPs). According to some embodiments, the drying includes lyophilization of the Silica-CysHex particles.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1—A schematic illustration of the final synthesis step to obtain the NP-CysHex particles (“SiO₂—N-CysHex), by binding CysHex peptides to functionalized silica NP (“SiO₂—N-maleimide”), according to some embodiments;

FIG. 2: Representative TGA thermogram for pristine Cab-O-Sil® M5 silica (N₂ atmosphere, heating rate 10° C./min). Mass loss in the range 25-125° C. is due to removal of absorbed water. Mass loss in the range 125-1000° C. attributed to water produced from silanol condensation;

FIG. 3A—Representative TGA thermograms of Cab-O-Sil® M5 silica ((“Pristine-SiO₂”), black), SiO₂—NH₂ (red), SiO₂-maleimide (blue) and SiO₂-CysHex (green). Experimental conditions: nitrogen atmosphere, heating rate 10° C./min;

FIG. 3B—Representative TGA of three overlayed thermograms of SiO₂-maleimide (N₂ atmosphere, heating rate 100° C./min);

FIG. 4A—Image of samples A-C; FIG. 4B-UV-VIS spectra of solutions containing silica NPs after treatment in NaOH solutions or dissolved in water. Silica in dionized water (cyan), 0.4M NaOH solution (blue), silica after treatment in 0.4M NaOH solution stirred at 45 C for 3 hr (red), silica after treatment in 0.5M NaOH solution stirred at 45 C for 18.5 hr (green);

FIG. 5A Chemical structure of APTMS molecule;

FIG. 5B—Chemical structure of dioxane molecule;

FIG. 5C—Chemical structure of trioxane molecule;

FIG. 5D—(d) H NMR (500 MHz) spectra of APTMS and dioxane dissolved in D2O (experiment 1, green); APTMS, untreated silica NP and dioxane after treatment in 0.4M NaOH/D2O (experiment 2, red); SiO2-NH2 and trioxane after silica dissolution in 0.4M NaOH/D2O (experiment 3, blue);

FIG. 6A-Chemical structure of N-propylmaleamic acid;

FIG. 6B-Chemical structure of N-propylmaleimide;

FIG. 6C—¹H NMR (400 MHZ) spectra of N-propylmaleamic acid (top spectrum-red) and N-propylmaleimide (bottom spectrum-blue) after treatment in 0.4M NaOH/D2O (experiment 2). Peak at 4.8 ppm corresponds to residual water. Peak at 5.2 ppm-trioxane external standard;

FIG. 7A—schematic illustration of silica nanoparticle functionalized by maleimide group (SiO₂-maleimide);

FIG. 7B H NMR (500 MHZ) spectrum of SiO₂-maleimide after particle dissolution in 0.4M NaOH/D2O (experiment 3). Peak at 5.2 ppm-trioxane external standard, peak at 4.8 ppm corresponds to residual water;

FIGS. 8A-8B—CysHex peptide ¹H NMR spectra: FIG. 8A—CysHex peptide dissolved in D2O (top curve, green-experiment 1), CysHex peptide dissolved in 0.4M NaOH/D2O (bottom curve, red-experiment 2); FIG. 8B—SiO₂-CysHex after silica dissolution in 0.4M NaOH/D20. Trioxane was used as an external standard (5.2 ppm). The regions marked “a” and “b” are used for peptide quantitative analysis;

FIG. 9—calibration curve of ¹H NMR unique peptide peaks group area as dependence on CysHex concentration in 0.4M NaOH solution. A sample with concentration of 1.40 (mg CysHex/ml NaOH solution) was used to check the accuracy of the calibration curves-marked with a star;

FIGS. 10—¹H NMR spectra of SiO₂—NH₂ (bottom-blue), SiO₂-maleimide (middle-red) and SiO₂-CysHex (top-green). Numbers correspond to peak assignments detailed in FIG. 5A, FIG. 7A and FIG. 8B. Trioxane peak at 5.2 ppm residual water at 4.8 ppm;

FIGS. 11—³¹P NMR (400 MHZ) spectra for 2.6 mM phosphate in FAS solution/D20. Spectrum at pH 10, phosphate peak at 2.6 ppm (top-green), pH 6.5 peak at 0.9 ppm (middle-red), and pH 4 peak at 0.2 ppm (bottom-blue). External standard, phosphoric acid, at 0 ppm. In phosphate/FAS solutions only the phosphate peak is observed;

FIGS. 12—³¹P NMR (400 MHZ) spectra of 2.6 mM phosphate (peak at 2.6 ppm) at pH=10 in FES solution/D2O (top-red), and FAS solution/D2O (bottom-blue). Phosphoric acid as external standard (0.0 ppm). Phospholipid peak observed at −0.7 ppm;

FIG. 13—Phospholipid peak width ratio in FES solution as function of pH. Squares-solution contains only peptide (13.1 mM), triangles-solution contains peptide (13.1 mM) and phosphate (2.6 mM). Phospholipid concentration in FES 2 mM;

FIG. 14—Phosphate peak half width ratio in FES (left) and FAS (right) solutions as function of pH. Peptide concentration 13.1 mM, phosphate 2.6 mM, sodium 200 mM, chloride 125 mM. The sample marked by triangle in the left plot corresponds to a solution with twentyfold lower NaCl concentration;

FIG. 15A—Image of samples for the calibration curve in a 96 well;

FIG. 15B—UV-VIS spectrum of a solution containing 0.015 mM phosphate in deionized water mixed with molybdenum blue reagents; and

FIG. 16 Percent of phosphate captured by SiO₂-CysHex as function of pH in intestinal and gastric fluid simulants. Initial phosphate concentration 0.15 mM. Peptide to phosphate molar ratio 5:1. Each data point is an average of 2-3 independent measurements.

### DETAILED DESCRIPTION OF THE INVENTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

The terms “polypeptide,” “peptide” and “protein” are used interchangeably herein to refer to a polymer of amino acid residues. The terms also apply to amino acid polymers in which one or more amino acid residue is an artificial chemical analogue of a corresponding naturally occurring amino acid, as well as to naturally occurring amino acid polymers. In some embodiments, one or more of amino acid residue in the peptide, can contain modification, such as but be not limited only to, glycosylation, phosphorylation or disulfide bond shape. Also provided are conservative amino acid variants of the peptides disclosed herein. Variants according to the invention also may be made that conserve the overall molecular structure of the encoded proteins or peptides. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved. As used herein, Amino acids and peptide sequences are marked using conventional Amino Acid nomenclature (single letter or 3-letters code). For example, amino acid “Serine” may be marked as “Ser” or “S” and amino acid “Cysteine” may be marked as “Cys” or “C”.

The term “construct”, as used herein refers to an artificially assembled or isolated nucleic acid molecule which may be comprises of one or more nucleic acid sequences, wherein the nucleic acid sequences may be coding sequences (that is, sequence which encodes for an end product), regulatory sequences, non-coding sequences, or any combination thereof. The term construct includes, for example, vectors, plasmids but should not be seen as being limited thereto. The term “regulatory sequence” in some embodiments, refers to DNA sequences, which are necessary to affect the expression of coding sequences to which they are operably linked (connected/ligated).

The nature of the regulatory sequences differs depending on the host cells. For example, in prokaryotes, regulatory/control sequences may include promoter, ribosomal binding site, and/or terminators. For example, in eukaryotes regulatory/control sequences may include promoters (for example, constitutive of inducible), terminators enhancers, transactivators and/or transcription factors. A regulatory sequence which is “operably linked” to a coding sequence is ligated in such a way that expression of the coding sequence is achieved under suitable conditions. In some embodiments, a “Construct” or a “DNA construct” refer to an artificially assembled or isolated nucleic acid molecule which comprises a coding region of interest and optionally additional regulatory or non-coding sequences. As used herein, the term “vector” refers to any recombinant polynucleotide construct (such as a DNA construct) that may be used for the purpose of transformation, i.e. the introduction of heterologous DNA into a host cell. One exemplary type of vector is a “plasmid” which refers to a circular double stranded DNA loop into which additional DNA segments can be ligated. Another exemplary type of vector is a viral vector, wherein additional DNA segments can be ligated into the viral genome. Certain vectors are capable of autonomous replication in a host cell into which they are introduced. The term “Expression vector” refers to vectors that have the ability to incorporate and express heterologous nucleic acid fragments (such as DNA) in a foreign cell. In other words, an expression vector comprises nucleic acid sequences/fragments (such as DNA, mRNA), capable of being transcribed or expressed in a target cell. Many viral, prokaryotic and eukaryotic expression vectors are known and/or commercially available. Selection of appropriate expression vectors is within the knowledge of those having skill in the art. The expression vectors can include one or more regulatory sequences.

As used herein, the term “transformation” refers to the introduction of foreign DNA into cells. The terms “transformants” or “transformed cells” include the primary transformed cell and cultures derived from that cell regardless to the number of transfers. All progeny may not be precisely identical in DNA content, due to deliberate or inadvertent mutations. Mutant progeny that have the same functionality as screened for in the originally transformed cell are included in the definition of transformants.

As used herein, the terms “introducing” and “transfection” may interchangeably be used and refer to the transfer of molecules, such as, for example, nucleic acids, polynucleotide molecules, vectors, and the like into a target cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, injection, and the like, or combinations thereof. The transfection of the cell may be performed on any type of cell, of any origin, such as, for example, human cells, animal cells, plant cells, and the like. The cells may be isolated cells, tissue cultured cells, cell lines, cells present within an organism body, and the like.

As used herein, the term “treating” includes, but is not limited to one or more of the following: abrogating, ameliorating, inhibiting, attenuating, blocking, suppressing, reducing, delaying, halting, alleviating or preventing symptoms associated with a condition. Each possibility represents a separate embodiment of the present invention. In some embodiments, the condition is a condition related to abnormal levels of phosphate. In some embodiments, the conditions is related to elevated levels of phosphate. In some embodiments, the condition is Hyperphosphatemia.

The terms “Hexa peptide”, “HexP” and “Hex” may interchangeably be used. The terms refer to a peptide having six amino acids, capable of binding phosphate (i.e., being a phosphate binder (phB). In some embodiments, the HexP has an amino acid sequence: Ser-Gly-Ala-Gly-Lys-Thr (SGAGKT) (SEQ ID NO: 1).

The term “Cys-Hex” is directed to a HexP having an additional cysteine amino acid at a terminal end thereof. In some embodiments, the Cys residue is at the N-terminus of the HexP. In some embodiments, a Cys-Hex peptide has an amino acid sequence Cys-SGAGKT (SEQ ID NO: 2). In some embodiments, the Cys residue serves as a linker amino acid, to allow linking/attaching/binding/reacting the HexP with silica nanoparticles.

The term “Silica” as used herein relate to various types of silica particles, at various sizes (for example, 1-1000 nm). In some embodiments, the silica particles are nanoparticles. The particles may be essentially spherical. In some embodiments, the silica nanoparticles can form aggregates or agglomerates (for example, in the range of 0.1-250 micrometers or any subranges thereof). In some embodiments, the silica may be selected from fumed silica, mesoporous silica, hollow silica spheres, precipitated silica, pyrogenic silica, and the like, Or any combination thereof. Each possibility is a separate embodiment.

The terms “Silica-pep”, “NP-pep”, SiO2-peptide”, “SiO2-CysHex PhB”, “NP-Cys-Hex” and “HexP-NP” may interchangeably be used. The terms relate to silica particles (for example, nanoparticles (NP)) attached/conjugated/bound to the HexP, in particular, via a Cys amino acid linker.

According to some embodiments, there is provided herein a novel non-systemic, pH-dependent, phosphate binder (PhB) particles, for the treatment of hyperphosphatemia patients. The particles include hexapeptide peptides grafted/attached/linked onto silica particles, targeted to bind phosphate in the GI tract. According to further embodiments, further provided herein are analytical tools for the quantitative chemical characterization of the synthesized particles. In some embodiments, as exemplified herein, the ability of the particles to capture phosphate was tested in simulants of GI fluids.

According to some embodiments, the preparation process of the peptide functionalized silica (SiO₂-CysHex) included two intermediate steps: synthesis of amine functionalized silica (SiO₂—NH₂) followed by its conversion into maleimide functionalized silica (SiO₂-maleimide).

According to some embodiments, as disclosed herein, a combination of methods was used to characterize the products of each synthesis step. Surprisingly, it was found that Elemental analysis (EA), a commonly used technique for quantitative determination of specific elements of relevance (such as nitrogen and sulfur), was found extremely inaccurate for these purposes. The analysis of the unbound CysHex peptide whose elemental composition is known, resulted in errors exceeding 20%, which rendered EA inappropriate. Thermogravimetric analysis (TGA) was found suitable for quantitative determination of mass changes as result of NP surface modification, however data analysis was complicated by the need to correct for mass loss as result of silanol degradation. Consequently, 1H qNMR method was utilized to characterize the maleimide and the peptide functionalization.

According to further embodiments, as exemplified herein, the ability of the peptide to bind phosphate as a function of pH was examined in GI fluid simulants. Initially, the free, unbound peptide ability to bind phosphate was studied by means of ³¹P NMR, at different pH values in small intestinal fluid simulants representing compositions related to fasted (FAS) and fed (FES) states. It was found that there is a competition on binding to the peptides between phosphate ions and the phospholipid molecules present in the intestinal fluids. This leads to lower phosphate-binding efficiency by this peptide sequence and requires higher peptide based PhB loading.

According to further embodiments, SiO₂-CysHex PhB efficiency was tested in the FAS, FES simulants at pH values of 2, 6, and 10. In addition, it was also tested in gastric fluid simulant (FEDGAS) at pH values of 3, 4.5, and 6, representing the late, middle, and early parts of meal digestion. For all three fluids the maximal phosphate binding was measured at pH of 6 which is the prevalent pH in the intestine. In the case of the gastric fluid pH=6 corresponds to the early stages of ingestion in the stomach. Hence, indicating that the phosphate binder should preferably be consumed with food.

According to some embodiments, as exemplified herein, the phosphate captured per gr of SiO₂-CysHex was calculated in intestinal fluid simulants and exhibited relatively high binding capacity of 2.6 and 1.5 mg of phosphate per 1 gr SiO₂-CysHex in FAS and FES solutions, respectively. According to some embodiments, in order to increase the binding efficiency, may include increasing the amount of peptide per silica particle. In some embodiments, increasing the length of the linker between the NP surface and the HEX peptide, to increase the distance of the peptide from the particle surface to alleviate steric constraints and allow for increased number of bound peptide molecules per particle.

According to some embodiments, there is thus provided a composition which includes silica nanoparticles conjugated to a phosphate binding hexapeptide (PBH) via a cysteine linker amino acid residue.

According to some embodiments, the average size of the silica nanoparticles may be in the range of about 1-500 nm, or any subranges thereof. In some embodiments, the average size of the silica nanoparticles may be in the range of about 2-300 nm. In some embodiments, the average size of the silica nanoparticles may be in the range of about 5-200 nm. In some embodiments, the average size of the silica nanoparticles may be in the range of about 6-100 nm. In some embodiments, the average size of the silica nanoparticles may be in the range of about 5-20 nm. In some embodiments, the average size of the silica nanoparticles may be in the range of about 7-15 nm. In some embodiments, the average size of the silica nanoparticles may be about 12 nm.

According to some embodiments, the silica nanoparticles may form aggregates and agglomerates, having a size in the range of about 0.1-250 micrometers, or any subranges thereof. In some embodiments, the silica aggregates may have a size in the range of about 0.2-200 micrometers.

According to some embodiments, the silica nanoparticles may have an average surface area in the range of about 50-600 m²/gr. According to some embodiments, the silica nanoparticles may have an average surface area in the range of about 100-400 m²/gr. According to some embodiments, the silica nanoparticles may have an average surface area in the range of about 150-350 m²/gr. According to some embodiments, the silica nanoparticles may have an average surface area in the range of about 180-220 m²/gr. According to some embodiments, the silica nanoparticles may have an average surface area of about 200 m²/gr.

According to some embodiments, each silica nanoparticle may be conjugated to about 1-400 Cys-Hex molecules. In some embodiments, each silica nanoparticle may be conjugated to about 50-350 Cys-Hex molecules. In some embodiments, each silica nanoparticle may be conjugated to about 100-300 Cys-Hex molecules. In some embodiments, each silica nanoparticle may be conjugated to about 150-250 Cys-Hex molecules. In some embodiments, each silica nanoparticle may be conjugated to at least about 50 Cys-Hex molecules. In some embodiments, each silica nanoparticle may be conjugated to at least about 100 Cys-Hex molecules. In some embodiments, each silica nanoparticle may be conjugated to at least about 150 Cys-Hex molecules. In some embodiments, each silica nanoparticle may be conjugated to at least about 180 Cys-Hex molecules. In some embodiments, each silica nanoparticle may be conjugated to at least about 200 Cys-Hex molecules. In some embodiments, each silica nanoparticle may be conjugated to at least about 250 Cys-Hex molecules. In some embodiments, each silica nanoparticle may be conjugated to about 200-225 Cys-Hex molecules.

According to some embodiments, the NP-Cys-Hex may bind phosphate at relatively high efficiency. In some embodiments, the binding efficiency may be affected according to PH levels and/or fasting state.

In some embodiments, the average phosphate binding efficiency of the NP-Cys-Hex may be in the range of about 1-7 mg phosphate (i.e., 1 gr particles can bind about 1-7 mg phosphate).

In some exemplary embodiments, the average amount of phosphate captured at pH=6 by 1 gr of particles may be about 1-3 mg (for example, about 2.6 mg) in FAS state (fasted state). In some exemplary embodiments, the average amount of phosphate captured at pH=6 by 1 gr of particles may be about 1-3 mg (for example, about 1.5 mg) in FES (fed state) state. In some exemplary embodiments, the average amount of phosphate captured at pH=6 by 1 gr of particles may be about 1-3 mg (for example, about 1.3 mg) in FEDGAS state (fed state gastric fluid).

According to some embodiments, the Hex-peptide disclosed herein may be produced by recombinant or chemical synthetic methods.

According to some embodiments, there is provided a composition (also referred to herein as pharmaceutical composition) which includes the NP-Cys-Hex particles. In some embodiments, the composition may include one or more suitable excipients, according to the purpose, type and/or use of the composition. In some embodiments, excipient is a pharmaceutical excipient which may include or a pharmaceutical carrier, vehicle, buffer and/or diluent.

In some embodiments, the composition disclosed herein may be used as a medicament for treating hyper phosphate related conditions, such as, hyperphosphatemia.

According to some embodiments, any suitable route of administration to a subject may be used for the particles or the composition of the present invention, including but not limited to, local and systemic routes. Exemplary suitable routes of administration include, but are not limited to: enterally, orally, naso-gastric, and the like. In some embodiments, administration routes to the GI tract are utilized.

According to another embodiment, administration systemically is through an enteral route. According to another embodiment, administration through an enteral route is buccal administration. According to another embodiment, administration through an enteral route is oral administration. According to some embodiments, the composition is formulated for oral administration. According to some embodiments, the composition is formulated for naso-gastric tubing.

According to some embodiments, oral administration is in the form of hard or soft gelatin capsules, pills, capsules, tablets, including coated tablets, dragees, elixirs, suspensions, liquids, gels, slurries, syrups or inhalations and controlled release forms thereof.

According to some embodiments, suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

In some embodiments, if desired, disintegrating agents, such as cross-linked polyvinyl pyrrolidone, agar, or alginic acid or a salt thereof, such as sodium alginate, may be added. Capsules and cartridges of, for example, gelatin, for use in a dispenser may be formulated containing a powder mix of the composition of the invention and a suitable powder base, such as lactose or starch.

According to some embodiments, solid dosage forms for oral administration include capsules, tablets, pill, powders, and granules. In such solid dosage forms, the composition of the invention is admixed with at least one inert pharmaceutically acceptable carrier such as sucrose, lactose, or starch. Such dosage forms can also comprise, as it normal practice, additional substances other than inert diluents, e.g., lubricating, agents such as magnesium stearate. In the case of capsules, tablets and pills, the dosage forms may also comprise buffering, agents. Tablets and pills can additionally be prepared with enteric coatings.

In some embodiments, liquid dosage forms for oral administration may further contain adjuvants, such as wetting agents, emulsifying and suspending agents, and sweetening, flavoring and perfuming agents. According to some embodiments, enteral coating of the composition is further used for oral or buccal administration. The term “enteral coating”, as used herein, refers to a coating which controls the location of composition absorption within the digestive system. Non-limiting examples for materials used for enteral coating are fatty acids, waxes, plant fibers or plastics.

According to some embodiments, the administration may include any suitable administration regime, depending, inter alia, on the medical condition, patient characteristics, administration route, and the like. In some embodiments, administration may include administration twice daily, every day, every other day, every third day, every fourth day, every fifth day, once a week, once every second week, once every third week, once every month, and the like.

According to some embodiments, the NP-Cys-Hex, may be used in combination with other therapeutic agents. The components of such combinations may be administered sequentially or simultaneously/concomitantly in separate or combined pharmaceutical formulations by any suitable administration route.

According to some embodiments, there is provided a method of treating hyperphosphatemia, the method includes administration to a subject in need thereof a therapeutically effective amount of the NP-Hex or a composition including the same.

According to some embodiments, a therapeutically effective amount refers to an amount sufficient to ameliorate and/or prevent at least one of the symptoms associated with hyperphosphatemia. In some embodiments, a therapeutically effective amount refers to an amount sufficient to reduce phosphate levels in a subject in need thereof.

According to some embodiments, there are provided kits comprising the herein disclosed nanoparticles and/or the composition as disclosed herein. Such a kit can be used, for example, in the treatment of hyperphosphatemia. In some embodiments, the kit may used in CKD patients.

According to some embodiments, there is provided a method for preparing the advantageous NP-Cys-Hex particles, the method includes one or more of the steps:
i) obtaining silica nanoparticles;
ii) substituting hydroxyl group(s) on the silica nanoparticles surface with amine group(s), to obtain modified (substituted) silica nanoparticles (“SiO₂—NH₂”).
iii) reacting maleic anhydride with the amine group(s) to yield maleimide end group(s) (“SiO₂-maleimide”); and
iv) binding/attaching/an hexapeptide having a cysteine amino acid end group linker to the maleimide functionalized nanoparticles, via said cysteine amino acid end group linker;
v) optionally, drying the NP-Cys-Hex particles

In some embodiments, dehydrated silica nanoparticles are used and may be suspended in toluene. Amine groups, including 3-aminopropyltrimethoxysilane (APTMS), are added to the silica mixture. The mixture may then be heated to the boiling point of toluene and stirred vigorously for a period of time (for example about 2 h) under nitrogen atmosphere. After colling and filtering the solution, the silica nanoparticles may be collected to obtain the substituted/modified silica nanoparticles (“SiO₂—NH₂”).

In some embodiments, for the functionalization of SiO₂—NH₂ into maleamic acid, maleic anhydride may be mixed with the modified silica nanoparticles and incubated for a period of time (for example, O/N). After washing excess maleic anhydride, the SiO₂-maleamic acid NPs are redispersed and recovered.

In some embodiments, for the synthesis of SiO₂-maleimide, the entire amount of the wet SiO₂-maleamic acid may be suspended in a solution. The suspension may be vigorously stirred and incubated at 90° C. for 2 h. After cooling, the SiO₂-maleimide is purified by centrifugation(s), redispersed in suitable solvents and recovered. The wet solid SiO₂-maleimide NP may further be dried.

According to some embodiments, binding/attaching/conjugating an hexapeptide having a cysteine amino acid end group linker to the maleimide functionalized nanoparticles may include the selective peptide attachment to the C═C bond of the maleimide via the SH group available on the side chain of cysteine. In some embodiments, the binding is facilitated in suitable buffers and under oxygen free inert atmosphere. In some embodiments, the peptide amount may be at an excess molar (for example, 2-fold molar excess) over the calculated maleimide groups. The resulting SiO₂-CysHex may be purified cycles of centrifugation and re-dispersed in a suitable liquid (such as, water). In some embodiments, the wet solid SiO₂-CysHex NP may be further frozen in liquid nitrogen and lyophilized.

In the description and claims of the application, the words “include” and “have”, and forms thereof, are not limited to members in a list with which the words may be associated. As used herein, the term comprising includes the term consisting of.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### Examples

### Example 1: Preparation of Silica-Cys-Hex Particles

Fumed silica NPs used were Cab-O-Sil® M5 (Cabot Corp, USA) with a single particle diameter of 12 nm and surface area of 200 m²/gr. The particles form branched chain aggregates with a length of approximately 250 nm, and 40 μm sized agglomerates.

Functionalization of Silica NP Surface with Amines (3-Aminopropyltrimethoxysilane ([(CH3O)3SiCH2CH2CH2NH2], APTMS):

5 g of dehydrated silica Cab-O-Sil® M5 were inserted into a 500 mL 3-necked flask fitted with a reflux condenser and CaCl₂) trap and connected to nitrogen supply. The silica was suspended in 70 mL of toluene (Bio-lab, 99.7%) and stirred vigorously with a mechanical stirrer. Next, 5 g of 3-aminopropyltrimethoxysilane (APTMS, Sigma-Aldrich, 97%) were added and the mixture was heated to the boiling point of toluene (b.p. 110-111° C.) using an oil bath (kept at 123° C.). The reaction was left to stir vigorously for 2 h under nitrogen atmosphere. The solution was cooled to room temperature (RT) and filtered using a funnel fitted with a glass frit P4 (10-16 μm pores) to collect the silica particles. The product, APTMS modified silica (SiO₂—NH₂) was washed with 500 mL toluene followed by 260 mL of methanol and dried in an oven for 1 h at 110° C. under vacuum yielding 4.35 g of modified silica.

Functionalization of Silica NP Surface with Maleimide (Maleamic Acid)

For the functionalization of SiO₂—NH₂ into maleamic acid, 400 mg maleic anhydride (MA, Sigma-Aldrich, 99.0%), and 0.5 g of SiO₂—NH₂ were mixed in 20 mL of glacial acetic acid (Bio-lab, 99.8%) and vigorously stirred overnight at RT. The excess of maleic anhydride was removed by centrifugation (4000 rpm, 10 min) and the NP were redispersed in 40 mL of 1:1 mixture of 2-propanol (DaeJung, 99.7%) and deionized water (DI-Millipore purified deionized water 18.2 MΩ*cm) followed by centrifugation, redispersion in 40 mL of ethanol, and recovery by centrifugation. The product was used wet without any further drying in the next step as described below.

For the synthesis of SiO₂-maleimide, the entire amount of the wet SiO₂-maleamic acid was suspended in a solution of 500 mg sodium acetate (Sigma-Aldrich, 99.0%) in 20 mL of acetic anhydride (Sigma-Aldrich, 99.0%). The suspension was vigorously stirred and kept at 90° C. for 2 h. The reaction mixture was cooled. SiO₂-maleimide was purified by two cycles of centrifugation (4000 rpm, 10 min) and re-dispersion in 40 mL of 1:1 mixture of 2-propanol (DaeJung, 99.7%) and DI water followed by centrifugation, redispersion in 40 mL of ethanol, and recovery by centrifugation.

Finally, the wet solid NP were dried in an oven for 1 h at 110° C. under vacuum yielding 0.35 g of the functionalized silica.

Functionalization of Silica NP Surface with CysHex:

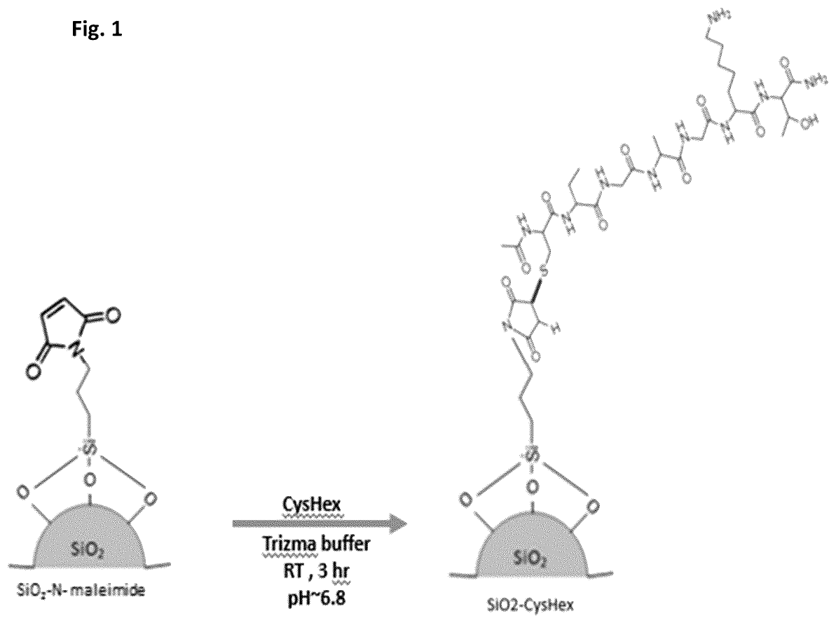

The final synthesis step to obtain CysHex peptide functionalized silica NP is shown schematically in FIG. 1.

Trizma® buffer composed of 2-Amino-2-(hydroxymethyl)-1,3-propanediol (Trizma® base 99.9%, Sigma-Aldrich) and Tris(hydroxymethyl)aminomethane hydrochloride (Trizma® hydrochloride 99.0%, Sigma-Aldrich) 0.25M background solution was used for selective peptide attachment to the C═C bond of the maleimide via the SH group available on the side chain of cysteine. Nitrogen was bubbled for 40 minutes into 40 mL Trizma® buffer in a 50 mL flask to secure oxygen free inert atmosphere. 130 mg of peptide with the sequence Cys-SGAGKT (CysHex, GenScript, Singapore) were inserted into a 10 mL flask reaction vessel placed on a magnetic stirrer hot plate and connected to nitrogen supply. This peptide amount corresponds to roughly 2-fold molar excess over the calculated maleimide groups. Trizma® buffer (˜6.7 mL) was added slowly to the flask until a pH of 6.7-6.8 was reached. Next, 200 mg of SiO₂-maleimide were added to the mixture, stirred vigorously with a magnetic stirrer, and kept at RT for 3 h. SiO₂-CysHex was purified by six cycles of centrifugation (4000 rpm, 20 min) and re-dispersion in 40 mL of DI water for 40 min followed by centrifugation and recovery by centrifugation. Finally, the wet solid NP were frozen in liquid nitrogen and lyophilized overnight.

### Example 2: Characterization of Silica NP-CysHex Synthesis Products

### Materials and Methods

XPS—XPS was employed to identify the different chemical species on the NP surface and determine their relative amounts. Data was collected using an X-ray photoelectron spectrometer ESCALAB 250 (Thermo Fisher Scientific) ultrahigh vacuum (1*10⁻⁹ bar) apparatus with an AlKα X-ray source and a monochromator. The X-ray beam size was 500 μm, survey spectra were recorded with pass energy (PE) of 150 eV, and high energy resolution spectra were recorded with PE of 20 eV. All spectra were calibrated relative to a carbon C 1s peak positioned at 284.8 eV to correct for charging effects. Processing of the XPS results was carried out using the AVANTAGE program (Thermo Fisher).

TGA—TGA model SDTA851 (Mettler-Toledo) was employed to quantify the mass addition on the NP after each synthesis step. In addition, TGA was used to determine the initial amount of hydroxyl groups on the silica NP surface. An amount of 8-10 mg of NP sample was inserted into a 70 μL TGA alumina crucible. The sample was heated from 25° C. to 1000° C. at a rate of 10° C.·min⁻¹ under a nitrogen atmosphere. To determine the surface concentration of silanol groups on the pristine silica particle, the silica sample was first compacted by pressing it into pellets (using a KBr pellet kit) under pressure (˜5 ton) for 5 minutes. The pellets were subsequently pulverized and inserted into the TGA crucible and heated as described above. TGA data was analyzed using STARe software (Mettler-Toledo).

¹H solution NMR—Quantitative ¹H NMR (1H qNMR) in solution, following particle dissolution in strong base, was used as the method of choice for quantifying functional groups attached to silica NPs. Kunc et al. (Anal. Chem. 2018, 90 (22), 13322-13330) describe an optimized H qNMR procedure for the determination of amine groups attached to silica NP.

The currently used procedure involved dissolution of the tested material in aq. NaOH followed by the ¹H NMR measurement as described in detail bellow. In each one of the measurement an external standard (either dioxane or trioxane) was added to allow quantitative determination of concentrations. For each of the three moieties to be analyzed (amine, maleimide, peptide) the following tests have been carried out: (i) ¹H NMR spectra of the individual reagents (e.g., APTMS, CysHex peptide) dissolved in D2O were obtained, all peak assignments identified, and relative magnitudes were compared to the expected values derived from their chemical structure. (ii) NMR spectra of the reagents dissolved in the NaOH solution under conditions identical to the ones used for the NP dissolution were obtained. It allowed determination of the effect of the dissolution procedure on the spectra in terms of peak shifts, shapes, and relative magnitudes. (iii) Step (ii) was repeated in the presence of pristine silica particles to identify the effect of dissolved silica fragments on the spectra. None was discovered. (iv) Finally, NMR spectra of functionalized silica following dissolution in the strong base were obtained, and surface concentrations were determined.

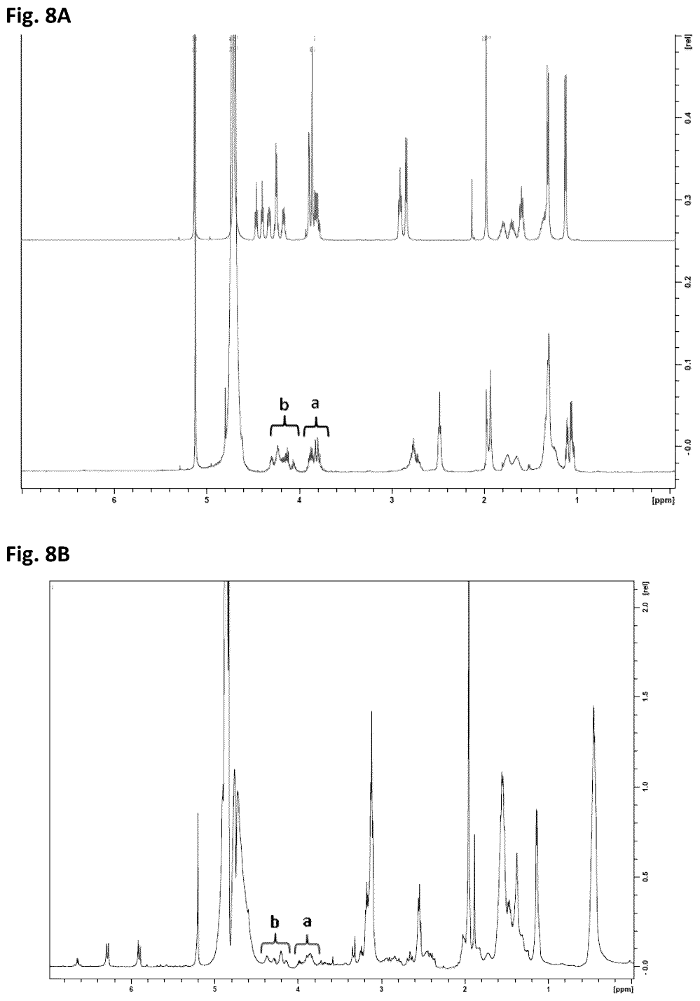

In addition, due to overlapping peptide peaks when dissolved in NaOH, as shown in FIGS. 8A-B, it was necessary to generate a calibration curve for peptide concentrations in NaOH to enable quantification of peptide surface attachment.

### Materials

Deuterium oxide (Sigma-Aldrich, 99.9%), Sodium hydroxide (Gadot), APTMS (Sigma-Aldrich, 97%), N-propylmaleimide (Sigma-Aldrich, 95%), N-propylmaleamic acid (Sigma-Aldrich, 99%), peptide with Cys-SGAGKT sequence (CysHex, GenScript, Singapore), 1,4-dioxane (Sigma-Aldrich, 99.8%), 1,3,5-trioxane (Sigma-Aldrich, >99%).

Sample Preparation for qNMR-D2O Solution

Reagents were weighed into 4 mL or 20 mL vials and D2O was added (amounts tabulated in Table 1). Dioxane or D2O solutions of trioxane were used as external standards.

TABLE 1
Sample composition for reagents
in D₂O for H qNMR measurements

| | | | | | Volume |
|---|---|---|---|---|---|
| | | | | Concen- | trioxane |
| | | | | tration | solution |
| | Volume | Mass of | Mass | trioxane in | added to |
| | D₂O | reagent | dioxane | D₂O | sample |
| | [mL] | [mg] | [mg] | [mM] | [μL] |
| APTMS | 1 | 1 | 1 | — | — |
| N- | 5 | 3.7 | — | 67 | 125 |
| propylmaleimide | | | | | |
| (95% purity) | | | | | |
| N- | 5 | 3.9 | — | 67 | 125 |
| propylmaleamic | | | | | |
| acid | | | | | |
| CysHex | 2 | 20 | — | 67 | 226 |

Sample Preparation for qNMR-Dissolution in NaOH

The protocol for measurements in presence of NaOH (items ii-iv above) consisted of weighing the reagents or the Silica NPs into 4 mL vials (amounts detailed in Table 2) followed by addition of 0.4M NaOH in D₂O while stirring with a magnetic stirrer. The sample was placed into an oil bath on a magnetic plate heated to 45° C. for 3 h. Heating the sample with vigorous stirring was necessary for complete dissolution of the NPs; otherwise, the aggregated NPs were observed to precipitate from the solution, resulting in incomplete hydrolysis. The sample was then removed from the heating plate and allowed to cool to RT. Trioxane in D₂O solutions (67 mM or 6.7 mM) was used as external standard. Finally, the vial was closed and vortexed for 5s. All relevant amounts for the different samples are provided in Table 2. The qNMR measurements were carried out within 24 h of sample preparation. Delayed measurements past 24 h occasionally resulted in peak shifting attributed to possible reactions between the dissolved organic components and the NaOH. Quantitative functional group concentrations were determined by comparing the areas under the trioxane peak and the average of areas under the selected resonance peaks for the components of interest.

TABLE 2
Sample composition of reagents or functionalized silica
NP for dissolution in NAOH and NMR experiments

| | Volume | Mass | Mass of | Concen- | |
|---|---|---|---|---|---|
| | 0.4M | pristine | reagent | tration | Volume |
| | NaOH in | silica | or silica | trioxane in | trioxane |
| | D₂O | NP | NP | D₂O | solution |
| | [mL] | [mg] | [mg] | [mM] | [μL] |
| APTMS | 10 | 100 | 17.9 | 13.5 mg * | — |
| SiO₂—NH₂ | 1 | — | 10 | 67 | 225 |
| N- | 5 | — | 3.7 | 67 | 125 |
| propylmaleimide | | | | | |
| (95% purity) | | | | | |
| N- | 5 | — | 3.9 | 67 | 125 |
| propylmaleamic | | | | | |
| acid | | | | | |
| SiO₂-maleimide | 1 | — | 10 | 67 | 50 |
| SiO₂-CysHex | 1 | — | 10 | 6.7 | 95 |

* In this sample weighed amount of trioxane powder was added directly into the sample.

Sample Preparation for qNMR-Calibration Curve for Peptide Dissolved in NaOH

Determination of peptide surface concentration was based on a calibration curve between the measured peptide peak areas and the peptide concentration in 0.4M NaOH solution. Use of the calibration curve was required due to difficulties in a direct assessment based solely on the external standard as demonstrated in FIG. 8. The samples for the calibration curve were prepared in the compositions listed in Table 3. Peptide stock solution was prepared with a concentration of 10 mg CysHex peptide per 1 mL of 0.4M NaOH solution in D₂O, and 6.7 mM trioxane in D₂O solution was used as external standard. The calibration curve was based on four samples spanning the range of possible peptide concentrations prepared as described herein. The upper limit of the calibration curve was selected to correspond to the highest possible concentration of peptide achieved theoretically by reacting all the maleimide groups available on the silica surface (1:1 molar ratio). A fifth solution was used to test the accuracy of the calibration curve. It yielded a concentration value within less than 10% from the expected value.

TABLE 3
Sample composition for calibration curve
of peptide in 0.4M NaOH solution

| | Volume | Volume | Volume |
|---|---|---|---|
| | NaOH | peptide | trioxane |
| | 0.4M in | stock added | solution added |
| Concentration [mg | D₂O | to sample | to sample |
| peptide/mL NaOH] | [μL] | [μL] | [μL] |
| 0.2 | 957 | 20 | 23 |
| 0.8 | 830 | 80 | 90 |
| 1.2 | 857 | 120 | 23 |
| 1.4* | 702 | 140 | 158 |
| 2 | 574 | 200 | 226 |

*Sample used to test the accuracy of the calibration curve.

¹H qNMR Experiment

Solution proton NMR spectra were collected on a Bruker Avance III 400 MHz and 500 MHz spectrometers (Bruker BioSpin, Germany). NMR data was analyzed using TopSpin software (Bruker). Run parameters varied between the different experiments based on signal optimization.

### Elemental Analysis

Elemental analysis carried out on Thermo Scientific Flash 2000 CHNS analyzer (Thermo Fisher Scientific, USA) was employed to determine the additional mass fraction of carbon, hydrogen, nitrogen, and sulfur on the NP surface following each synthesis step. The samples were prepared by weighing 2-3 mg in a tin capsule, sealed, and inserted to the combustion reactor.

### Phosphate Binding in Intestinal and Gastric Fluid Simulants

GI fluid simulants were used to mimic the fluids present in the small intestine in the fasted and fed states and in the stomach after a high-fat FDA meal. It allowed examining the effects of physiological compositions and pH on the ability of the peptide-based particles to bind phosphate.

### GI Fluid Simulants—(FAS, FES, and FEDGAS)

The fluid simulants used in this study (Biorelevant® media fluids, from Biorelevant, UK), included the fasted state simulated intestinal fluid version 2 (FaSSIF-V2 or FAS), fed state simulated intestinal fluid version 2 (FeSSIF-V2 or FES), and fed state simulated gastric fluid (FEDGAS).

The simulants compositions are shown in Table 4.

TABLE 4
Composition of FaSSIF-V2, FeSSIF-V2 and FEDGAS
solutions (according to manufacturer).

| | Simulant composition | | |
|---|---|---|---|
| | FaSSIF-V2 | FeSSIF-V2 | FEDGAS |
| Component | [mM] | [mM] | [g/L] |
| Taurocholate | 3 | 10 | — |
| Phospholipids | 0.2 | 2 | — |
| Sodium | 106 | 218 | — |
| Chloride | 69 | 125 | — |
| Maleic acid | 19 | 55 | — |
| Oleate | — | 0.8 | — |
| Glycerol | — | 5 | — |
| monooleate | | | |
| Fats | — | — | 69.4 |
| Carbohydrates | — | — | 69.4 |
| Dietary fiber | — | — | 3.4 |
| Bile salts | — | — | 0.3 |

### Preparation of GI Fluid Simulants

Solutions were prepared following the instructions form the manufacturer. FAS solutions were prepared at a ratio of 1.79 gr FAS powder per 1 L of 10% wt D₂O/H₂O. FES solutions were prepared at a ratio of 9.76 gr FES powder per 1 L 10% wt D₂O/H₂O. FEDGAS solutions were prepared at a ratio of 208.84 gr FEDGAS gel per 1 L DI H₂O. The solutions were prepared by mixing the powders or gel with the solvents. FAS and FES solutions could be used within 48 hrs, and the FEDGAS solution could be used within 24 hrs. The buffer solution supplied by the manufacturer to adjust the pH to the relevant physiological value has not been used here and pH values were adjusted as discussed below.

### Fluid Simulants Dilution

NaCl was found to interfere with the phosphate binding to peptides. In view of the relatively high chloride concentrations in the different simulants (cf. Table 4) it was necessary to adjust the concentration of the solutions to maintain a phosphate to NaCl concentration ratios similar to the ones found in the GI tract. The intake phosphate concentration in the GI tract following one meal is between 50-100 mM and the concentration of sodium is 200 mM. Due to experimental limitations, the concentration of phosphate used in the NMR experiments described below is 2.6 mM and for the colorimetric experiments 0.15 mM. As result, FAS and FES solutions were diluted by a factor of 20 for the NMR experiments and by a factor of 350 for the colorimetric experiments.

³¹P NMR-NMR relaxation is the process by which excited magnetic state nuclei return to their equilibrium distribution. At the start of an NMR experiment, the nuclei are “lined up” with the external magnetic field. When an electromagnetic RF field is activated on these nuclei, they resonate at a characteristic resonance frequency, and the NMR signal is created. At that instant, all the nuclei are in phase and coherent. However, due to small differences in their resonance frequencies, they will begin to dephase. The time it takes for the nuclei to dephase is measured by T₂ the so-called spin-spin relaxation time. T₂ is inversely proportional to Δω, the width of the resonance peak in the spectra measured at half the maximum height: Δω=2/T₂. The more slowly a macromolecule tumbles, the shorter its T₂; thus, big molecules have broader resonance peaks than small molecules. An increase in molecular size due to interaction or reduced mobility affects the T₂ relaxation time and is manifested by widening the NMR signal.

In this study, phosphate-binding experiments of free (unattached to silica) peptides in intestinal fluid simulants as function of the surrounding pH were examined. Binding was determined by the widening of the relevant phosphate peaks in phosphorus NMR (³¹P NMR) spectra. These experiments aimed to investigate the effect of the background solution containing many components on the ability of the peptide to bind phosphate. ³¹P NMR experiments were preferred over standard ¹H NMR since in the former only one (phosphate) or at most two (phosphate and phospholipid) peaks are detected as opposed to the multitude of frequently overlapping, peaks in the spectra of the latter.

### Chemicals:

Deuterium oxide (Sigma-Aldrich, 99.9%), FeSSIF-V2 and FaSSIF-V2 (Biorelevant), Sodium phosphate dibasic (Sigma-Aldrich, 99.0%), CysHex peptide (sequence SGAGKT-Hex, GenScript), phosphoric acid (Bio lab), Solvent used 10% wt D₂O/H₂O. NaOH and HCl solutions of 2M, 1M, 0.5M and 0.1M were used for pH adjustment.

### Sample Preparation

Each ³¹P NMR experiment involved a comparison between two separate samples in either FAS or FES solution at a specific pH value. The first sample contained only a phosphate and in the second, the peptide was added with a 5:1 molar excess over the phosphorus element. In addition, the interaction between the peptide and the phospholipid present in large amount in the FES solution, was also examined in the absence of a phosphate.

For these experiments, two stock solutions were prepared: phosphate stock-250 ppm of anhydrous Na₂HPO₄ in FAS or FES solution, and peptide stock-10 mg peptide in 1 mL FAS/FES solution (or 104 ppm peptide).

### Sample Preparation for the Phospholipid-Peptide Interaction Experiments

The first sample used for FES solution reference was composed of 1 mL FES solution which was placed in a 1.5 mL Eppendorf tube. The second sample used to study the phospholipid peptide interaction contained 874 μL peptide stock and 126 μL FES solution placed in another 1.5 mL Eppendorf tube. The pH was measured using a pH electrode (Mettler Toledo, SevenComplex) and adjusted to the desired value by dropwise addition of HCl or NaOH solutions. The two Eppendorf tubes were sealed and vortexed for 5 seconds. The contents of each Eppendorf tube were analyzed separately by NMR as described below.

### Sample Preparation for the Phosphate-Peptide Interaction Experiments

Samples were prepared by mixing the required amounts of phosphate stock solution, and FAS or FES solutions into one 1.5 mL Eppendorf tube, and phosphate stock, peptide stock, and FAS or FES solution into a second similar tube. in. The amounts used for each experiment are provided in Table 5. The pH was measured and adjusted to the desired value as described above. The Eppendorf tubes were sealed and vortexed for 5 seconds. The contents of each Eppendorf tube were analyzed separately by NMR as described below.

TABLE 5
Sample compositions for ³¹P NMR experiments

| | Volume | Volume | Volume |
|---|---|---|---|
| | phosphate | peptide | FAS/FES |
| | stock [μL] | stock [μL] | solution [μL] |
| Phosphate | 125 | 0 | 875 |
| Phosphate and | 125 | 874 | 1 |
| peptide | | | |

### ³¹P NMR Experiment

Solution ³¹P NMR data were collected on a Bruker Avance III 400 MHz spectrometer (Bruker BioSpin, Germany). The relaxation delay was 3 s and the number of scans was 120. A capillary tube insert filled with phosphoric acid solution in D₂O (10% v/v phosphoric acid in D₂O/H₂O) was used for calibration and inserted into the NMR tube that contained the measured sample. Since the evaluation of the results was based on comparison of resonance peak widths obtained from different samples, identical phosphoric acid peak size in the different experiments was used as an indication for identical test conditions validating the comparison. NMR data was analyzed using TopSpin software (Bruker).

### Colorimetric Method

The binding efficiency of the SiO₂-CysHex particles in intestinal and digestive fluid simulants was examined by a colorimetric method. The procedure was based on the determination of the amount of uncaptured phosphate from a solution of known initial phosphate concentration. A spectrophotometric assay referred to as the molybdenum blue reaction was employed. It emits a blue color whose intensity is linearly proportional to the amount of phosphate present in the solution allowing by measurement of color intensity and proper calibration, to determine phosphate concentration. The reaction involves the following two steps:
1. PO₄³⁻+12(NH₄)₂MoO₄+24H⁺→(NH₄)₃PO₄·12MoO₃+21NH₄⁺+12H₂O
2. (NH₄)₃PO₄·12MoO₃+reductant→molybdenum blue

For this experiment, a phosphate assay kit (Sigma-Aldrich) with a stated concentration range between 0.005 mM and 0.025 mM phosphate was used. The kit included a 10 mM phosphate standard solution and a coloring reagent that reacts with phosphates to form a complex that could be measured and quantified by UV-VIS absorbance spectroscopy. The phosphate standard was used to prepare a calibration curve between the absorbance intensity at a wavelength of 650 nm and phosphate concentrations.

### Binding Experiment

Chemicals: FeSSIF-V2, FaSSIF-V2 and FEDGAS (Biorelevant), and Sodium phosphate dibasic (Sigma-Aldrich, 99.0%), water (Millipore purified deionized (DI) water 18.2 M2*cm).

### Solutions

FAS solution was prepared at a ratio of 1.79 gr FAS powder per 1 L DI water. FES solution was prepared at a ratio of 9.76 gr FAS powder per 1 L DI water as listed in the preparation instructions from Biorelevant. Next, the solutions were diluted by 350 to maintain the same ratio between phosphate to NaCl in the FAS and FES solutions as explained in sec. 4.3.1.1.1 above. FEDGAS solution was prepared by mixing FEDGAS gel and DI water at a ratio of 170 gr FEDGAS gel per 1 L DI water and diluted by a factor of 350 to match the dilution of FAS and FES solutions. Anhydrous Na₂HPO₄ (0.15 mM) was added to the diluted FAS/FES/FEDGAS solutions. Solutions were tested immediately following preparation.

### Sample Preparation for the Binding Experiments

A volume of 1 mL of 0.15 mM phosphate GI simulant solution was combined in an Eppendorf tube with a predetermined mass of SiO₂-CysHex to achieve a molar ratio of CysHex:phosphate of 5:1. Upon combining the silica NP and phosphate solution, the pH was adjusted to the desired pH value by adding dropwise HCl or NaOH solutions while vortexing between additions. The pH was measured using an electrode-based pH meter (SevenComplex, Mettler Toledo). Upon reaching the target pH, the suspension was rotated on a vial rotator for 40 minutes to allow good contact between the peptide and the phosphate molecules. Lastly, the suspension was centrifuged at 4000 rpm for 15 minutes, and the supernatant was filtered using a 13 mm 0.22 μm Axiva syringe filter into another Eppendorf tube.

### Absorbance Reading

Chemicals: phosphate assay kit (Sigma-Aldrich) which contained 10 mM phosphate standard solution and the reagent mixture to form molybdenum blue.

Instrumentation: absorbance spectroscopy data was collected on Tecan Infinite 200 plate reader (Mannedorf, Switzerland).

### Calibration Curve:

To conduct the assay for the calibration curve samples, 0.1 mM phosphate stock solution was prepared by mixing 10 μL of 10 mM phosphate standard provided by the kit and 990 μL of DI water. A set of solutions of varying phosphate concentrations in the range from 0 to 0.025 mM, was obtained by adding the appropriate amounts of DI water and 0.1 mM phosphate solution to a clear bottom 96 well plate as detailed in Table 6. Lastly, 30 μL of coloring reagent was added to each well and allowed to react for 30 minutes. The plate was subsequently placed in the spectrophotometer and the absorbance was measured at a wavelength of 650 nm. Each time the colorimetric method was employed to determine the unknown phosphate concentration in a tested solution, an additional 0.015 mM standard sample was measured as well to revalidate the calibration curve.

TABLE 6
Calibration curve for UV-VIS absorbency as a dependance
on phosphate concentration

| Phosphate | Volume 0.1 mM | |
|---|---|---|
| concentration | phosphate stock | Volume DI water |
| [mM] | [μL] | [μL] |
| 0 | 0 | 200 |
| 0.005 | 10 | 190 |
| 0.010 | 20 | 180 |
| 0.015 | 30 | 170 |
| 0.020 | 40 | 160 |
| 0.025 | 50 | 150 |

Absorbance Reading for Filtrate after Binding Experiment:

A volume of 20 μL of the supernatant filtrate (sec 4.3.3.1.2), 180 μL DI water, and 30 μL coloring reagent were added to a 96 well plate. Two duplicates of each sample were measured, and the resulting values were averaged. The samples in the plate were allowed to react for 30 minutes.

Afterward, the plate was placed in the spectrophotometer, and the absorbance was measured at a wavelength of 650 nm.

### Clinically Employed PhB—Sevelamer Carbonate and Lanthanum Carbonate

Chemicals: Sevelamer carbonate (Renvela®, Sanofi, France), Lanthanum carbonate (Fosrenol®, Shire Pharmaceuticals, UK), FeSSIF-V2 and FaSSIF-V2 (Biorelelant, UK), phosphate assay kit (Sigma-Aldrich) and Sodium phosphate dibasic (Sigma-Aldrich, 99.0%).

Phosphate binding efficiency experiment were conducted using sevelamer carbonate and lanthanum carbonate, the two most commonly used phosphate binders in clinical practice for the treatment of hyperphosphatemia patients. Experiments were performed at pH=7.5 in FAS or FES solutions. A volume of 5 mL of 0.15 mM phosphate solution was combined with 2.5 mg of Renvela® or Fosrenol® and phosphate binding experiments were carried out as described above.

### Phosphate Binding Efficiency was Measured by the Colorimetric Method as Described Above.

Determination of the surface silanol group concentration for Cab-O-Sil® M5 The surface concentration of silanol groups for Cab-O-Sil® M5 silica NP was determined by TGA analysis. For non-porous silica as is the case here, physisorbed water is removed at temperatures below 125° C. The three types of silanol groups encountered on fumed silica surfaces all undergo condensation at different temperatures between approximately 125° C. and 1000° C. The condensation occurs by the reaction between two silanol groups resulting in the release of one water molecule and the formation of one siloxane bond. Assuming no other groups other than water are released from the sample, and all silanol groups have undergone condensation, the amount of surface hydroxyl groups may be estimated from the mass loss obtained by the TGA measurement and eq 5.1.

### Results

### Characterization of Silica Nanoparticles Surface:

### Determination of the Surface Silanol Group Concentration for Cab-O-Sil® M5

The surface concentration of silanol groups for Cab-O-Sil® M5 silica NP was determined by TGA analysis. For non-porous silica as is the case here, physisorbed water is removed at temperatures below 125° C. The three types of silanol groups encountered on fumed silica surfaces all undergo condensation at different temperatures between approximately 125° C. and 1000° C. The condensation occurs by the reaction between two silanol groups resulting in the release of one water molecule and the formation of one siloxane bond. Assuming no other groups other than water are released from the sample, and all silanol groups have undergone condensation, the amount of surface hydroxyl groups may be estimated from the mass loss obtained by the TGA measurement and eq 5.1 as follows:

$${n}_{OH} = 2 {n}_{{H}_{2} O} = 2 ( \frac{WL ( 1000 ° C . ) - WL ( 125 ° C . )}{100 {Mw}_{{H}_{2} O}} ) \qquad Eq 5.1$$

Where n_{OH} is the number of moles of surface hydroxyl groups per gram of silica, n_{H}_{₂}_{O} is the number of moles of water released per gram of silica, WL(1000° C.)-WL(125° C.) is the measured weight loss (% wt) in the temperature range of dehydroxylation, and Mw_{H}_{₂}_{O} is the molar mass of water.

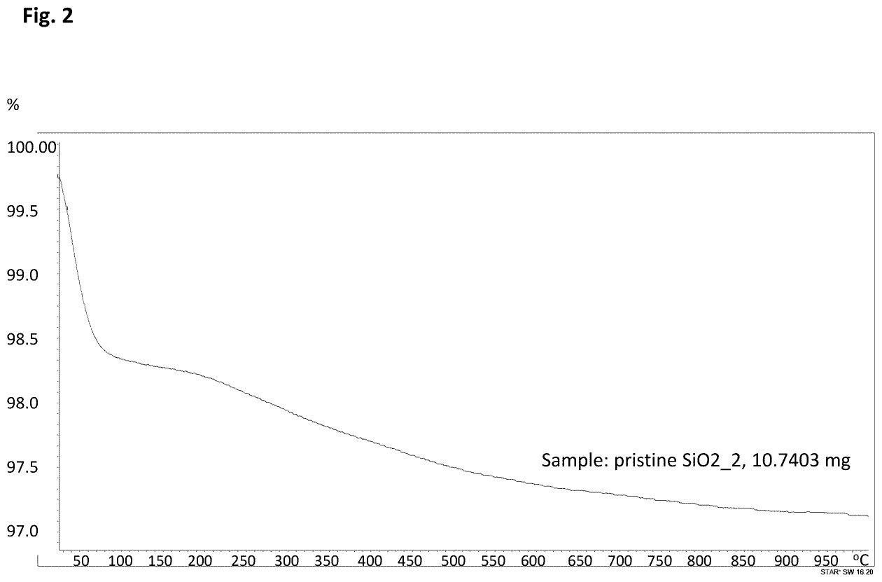

The measurements carried out under nitrogen atmosphere at a heating rate of 10° C./min yielded a weight loss of 1.34±0.12% over the relevant temperature range (cf. the thermogram in FIG. 2), which corresponds to 1.49±0.13 mmols of OH groups per gram of silica. With particle surface area of 200 m²/g³⁷ it amounts to 4.48±0.40 OH groups/nm², in agreement with literature values of 3.7-5.2 OH/nm²

XPS Analysis of Silica Surface Modification with APTMS

The amine functionalization of the silica surface with APTMS was confirmed by XPS analysis. This modification should result in the addition of nitrogen atoms to the silica surface. If all methoxy groups of APTMS react the ratio of C atoms to N atoms should be 3:1. Since it is reasonable to expect that not all methoxy groups will actually react with surface silanols the presence of the unreacted methoxy groups will result in a C/N ratio of either 4:1, or 5:1 for the case of bi- or uni-lateral binding of APTMS to the surface silanols, respectively. Table 7 provides the relative amounts of different atoms detected on the silica surface after modification with APTMS. The measured C/N ratio for the APTMS modified silica is 4.2. For pristine silica, only silicon, oxygen, and carbon atoms were detected. The presence of carbon is attributed to CO₂, which is known to adsorb to the silanol groups on the silica surface and even more so to amine-functionalized silica. Correcting the relative amounts of C atoms on the APTMS modified silica to account for adsorbed CO₂ based on the amount of CO₂ absorbed on the pristine silica results in a C/N ratio of 3.6. Thus, both values (corrected and uncorrected) of approximately 4:1 (Table 7) indicate that on average, two out of the three methoxy groups of the APTMS molecule have reacted.

In Table 7: The values in parentheses represent values corrected for absorbed CO₂ based on the value for pristine silica. The values for the pristine silica and SiO₂—NH₂ are an average of two or three different samples, respectively, obtained from the same synthesis batch.

TABLE 7
Relative atomic abundance detected by XPS on the surface of APTMS-modified silica.

| | Si | C | N | O | C/N |
|---|---|---|---|---|---|
| Sample | (atomic %) | (atomic %) | (atomic %) | (atomic %) | (atomic ratio) |
| Pristine SiO₂ | 35.9 ± 0.3 | 1.7 ± 0.1 | 0 | 62.4 ± 0.2 | — |
| SiO₂—NH₂ | 30.4 ± 0.2 | 12.2 ± 0.4 | 2.9 ± 0 | 54.5 ± 0.6 | 4.2 ± 0.1 |
| | | (10.5 ± 0.5) | | | (3.6 ± 0.2) |

TGA Analysis of Silica Surface Modification with APTMS, Maleimide and CysHex

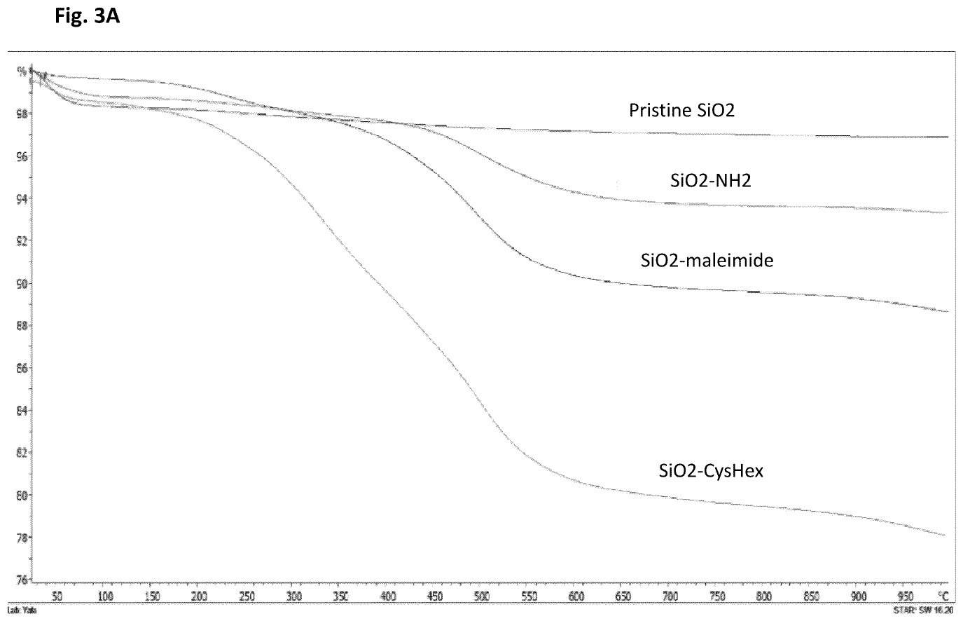

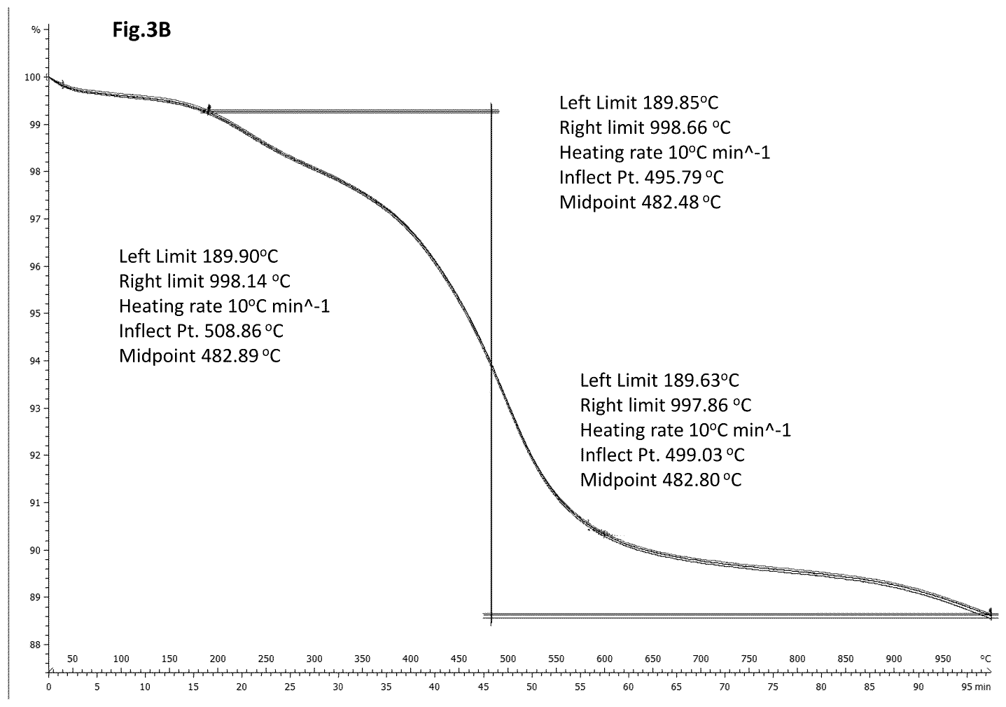

The amount of material successfully attached to the silica surface by the different functionalization steps was obtained by TGA analysis. Representative thermograms for pristine Cab-O-Sil® M5 silica (“Pristine Silica”), SiO₂—NH₂, SiO₂-maleimide and SiO₂-CysHex are depicted in FIG. 3A. Three samples were measured for each material apart from the peptide functionalized silica for which only one measurement was carried out. TGA measurements for the different samples were highly reproducible as can be observed by a representative three overlayed thermograms for SiO₂-maleimide (FIG. 3B).

The functionalized silica particles show an initial mass loss at low temperatures attributed to residual solvents or reactants. The mass of material successfully attached to the silica surface as result of the different functionalization steps was determined from the mass loss in the temperature range of 190-1000° C. The results are presented in Table 8.

In Table 8, percent of mass lost by heating the functionalized silica over the temperature range of 190-1000° C. is presented. For pristine silica, SiO₂—NH₂ and SiO₂-maleimide values represent average from three different samples.

TABLE 8
TGA results

| sample | % wt loss |
|---|---|
| Pristine silica | 1.34 ± 0.12 |
| SiO₂—NH₂ | 5.34 ± 0.02 |
| SiO₂-maleimide | 10.66 ± 0.01 |
| SiO₂-CysHex | 19.85 |

The values in Table 8 need to be corrected to account for mass loss due to condensation of unreacted silanol groups occurring over the same temperature range. Since the exact amount of unreacted silanols is unknown, an upper limit (all mass loss is associated with the addition of functional groups) and lower limit (the mass loss for pristine silica over the temperature range 190-1000° C. is subtracted) are calculated and presented in Table 9 in terms of the number of functional groups per nm². In order to obtain the surface coverage from the mass loss during the TGA experiment assumptions are made regarding the size of the molecular fragment detached from the silica surface upon thermal degradation. The detailed calculations include:

To obtain the surface coverage from the mass loss during the TGA experiment the following equation is used:

$$\frac{\# functional groups}{{nm}^{2}} = \frac{{( WL ( 1000 °C ) - WL ( 190 °C ) )}_{sub}}{100 {Mw}_{functional group}} * \frac{{N}_{A}}{{SA}_{silica}} \qquad ( C \text{.1} )$$

(WL(1000° C.)-WL(190° C.))_{sub} is the measured weight loss (% wt) between 190° C. and 1000° C. after subtracting the weight loss of all the previous synthesis steps, N_{A} is Avogadro number, and SA_{silica} is the surface area of a single silica NP (2*10²⁰ nm²/gr). Mw_{functional group} is the molar mass of the investigated functional group. The exact bond at which the APTMS molecule disconnects from the silica NPs surface upon thermal degradation is not determined, but it is believed that the breakup occurs at the bond between the silicon and the first carbon in the carbon chain and not between the silicon and the oxygen. This assumption is based on the lower Si—C bond energy (318 KJ/mol) in comparison to Si—O (452 KJ/mol).

The efficiency of the NH₂ groups attached to the silica surface obtained by the TGA is in good agreement with the XPS evaluation. As described in the previous section, on average two out of the three methoxy groups of the APTMS molecule have reacted with the hydroxyl groups on the silica surface. The initial number of OH groups per nm² is 4.5, therefore, the estimated number of NH₂ groups should be approximately 2.25, which is within the range of values obtained by the TGA as shown in Table 9.

The next functionalization step should yield one maleimide group per every amine group. Yet, based on the TGA results only approximately half of the amine groups have been converted into maleimide groups despite the large excess of maleic anhydride reactant. The low conversion of this reaction is possibly due to the steric limitations of the bulky maleamic acid and maleimide groups.

Similarly, based on the stoichiometry of the reaction it is expected, that the number of CysHex groups attached will be equal to the number of maleimide groups. However, it is found to be lower by a factor of 3 as shown in Table 9. This again is attributed to steric effects that may prevent achieving higher reaction conversion as discussed below.

TABLE 9
The number of functional groups attached to the silica surface following each
synthesis step as calculated from TGA mass loss in the temperature range of
190-1000° C. Upper and lower limit values provided.

| | Upper limit | Lower limit |
|---|---|---|
| Sample | (# groups/nm²) | (# groups/nm²) |
| SiO₂—NH₂ | 2.77 ± 0.01 | 2.18 ± 0.01 |
| SiO₂-maleimide | 1.51 ± 0.01 | 1.19 ± 0.01 |
| SiO₂-CysHex | 0.42 | 0.36 |

Approximately 20% difference is observed between the upper and lower limit values in Table 9. Due to this large uncertainty, another analytical method has been applied namely, ¹H quantitative NMR (¹H qNMR). It is aimed at reducing the uncertainty in the determination of the effectiveness of the different functionalization steps.
¹H qNMR Analysis of Silica Surface Functionalization with APTMS, Maleimide and CysHex

Kunc et al. have described a method for the determination of the number of amine groups on functionalized silica by means of liquid ¹H NMR. The method is based on dissolution of the silica particles in sodium hydroxide, rendering the functional groups accessible to liquid NMR. The authors claim this procedure offers a considerably more accurate means for the determination of the surface density of the amines relative to TGA. Herein, the method was extended to the determination of maleimide and CysHex as well.

The number of moles of a functional group attached to the silica surface (n_{functional group}) was determined from the areas under a relevant NMR peak and under the peak corresponding to the external standard, (dioxane or trioxane) as follows:

$${n}_{functional group} = \frac{{I}_{j}}{\# {H}_{j}} * \frac{\frac{{m}_{standard}}{{MW}_{standard}}}{\frac{{I}_{standard}}{\# {H}_{standard}}} \qquad Eq . 5.2$$

Where j is a selected proton belonging to the functional group, #H is the number of protons of type j or the standard, I is the integral under the peak assigned to proton type j or the standard, m_{standard} is the mass of the standard [gr], and MW_{standard} is the molar mass of the standard [gr/mol]. An example for this calculation is shows below:
H NMR signals of functionalized NPs
APTMS IN D₂O

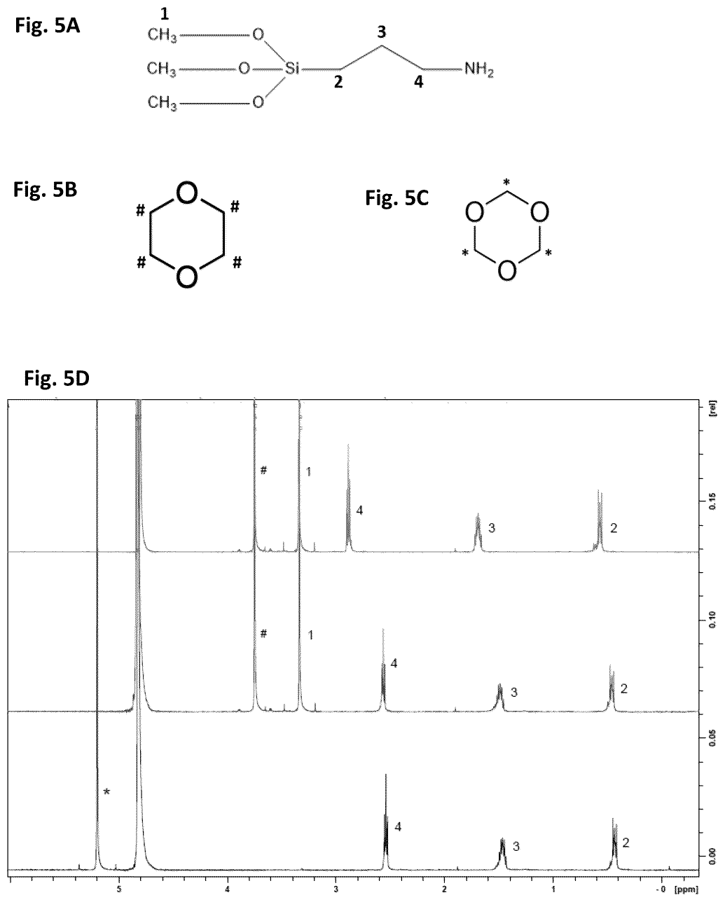

TABLE 10
H NMR signals and results for APTMS in D₂O.

| Peak label in FIG. 5 | Dioxane | 1 | 4 | 3 | 2 |
|---|---|---|---|---|---|
| Peak location [ppm] | 3.6 | 3.2 | 2.5 | 1.4 | 0.3 |
| Number of protons | 8 | 9 | 2 | 2 | 2 |
| Integral of NMR peak | 8 | 8.758 | 1.975 | 1.972 | 1.970 |
| mmol APTMS from NMR | — | 0.195 | 0.198 | 0.198 | 0.197 |

The averaged mmol APTMS from groups 1-4 obtained by H NMR is 0.197 mmol. The known mmol of APTMS in the sample was 0.200 mmol.

The accuracy of the NMR measurements was determined as follows:

$$\% error = ( \frac{{n}_{known} - {n}_{H NMR}}{{n}_{known}} ) * 100 \% = ( \frac{0.2 - 0.197}{0.2} ) * 100 \% = 1.5 \%$$

Where n_{H NMR} refers to the mmols obtained from the H NMR integration and n_{known} is the known amount of mmols APTMS added to the sample.

APTMS Mixed with Silica NP after Treatment in 0.4M NaOH:

TABLE 11
H-NMR signals and results for APTMS mixed with silica
NP (not attached) in 0.4M NaOH

| Peak label in FIG. 5 | Trioxane | 1 | 4 | 3 | 2 |
|---|---|---|---|---|---|
| Peak location [ppm] | 5.2 | 3.2 | 2.5 | 1.4 | 0.3 |
| Number of protons | 6 | 9 | 2 | 2 | 2 |
| Integral of NMR peak | 6 | 6.076 | 1.309 | 1.309 | 1.295 |
| mmol APTMS from NMR | — | 0.100 | 0.097 | 0.097 | 0.096 |

The averaged mmol APTMS from groups 1-4 obtained by H NMR is 0.097 mmol. The number of mmol of APTMS introduced into the sample was 0.100 mmol corresponding to 3.0% error.

SiO₂—NH₂ after Treatment in 0.4M NaOH:

TABLE 12
H NMR signals and results for SiO₂—NH₂ after
dissolution in 0.4M NaOH

| Peak label in FIG. 5 | Trioxane | 4 | 3 | 2 |
|---|---|---|---|---|
| Peak location [ppm] | 5.2 | 2.9 | 1.4 | 0.3 |
| Number of protons | 6 | 2 | 2 | 2 |
| Integral of NMR peak | 6 | 1.154 | 1.201 | 1.188 |
| mmol APTMS from NMR | — | 0.009* | 0.010 | 0.010 |

*Example of the calculation of the number of mmols of APTMS attached using eq. 5.2: Mass of trioxane used was 1.45 mg.

$${n}_{APTMS} = \frac{{I}_{4}}{\# {H}_{4}} * \frac{\frac{{m}_{trioxane}}{{MW}_{trioxane}}}{\frac{{I}_{trioxane}}{\# {H}_{trioxane}}} = \frac{1.154}{2} * \frac{\frac{1.45 mg}{\frac{90.08 mg}{m mol}}}{\frac{6}{6}} = 0.009 m mol APTMS$$

The number of mmol APTMS from the averaged results for groups 2-4 was 0.010 mmol. To convert the number of mmol functional groups to the number of functional groups (such as amines) attached per nm² eq. 5.3 used:

$$\frac{\# functional groups}{{nm}^{2}} = \frac{{n}_{H NMR}}{{m}_{SiO 2 - functional group}} * \frac{{N}_{A}}{{SA}_{silica}} = \frac{0.01 m mol}{9.89 mg} * \frac{6.02 * {10}^{20} \# groups / n mol}{2 * \frac{{10}^{17} {nm}^{2}}{mg}} = 2.9 \qquad Eq . 5.3$$

N-Propylmaleimide after Treatment in 0.4M NaOH:

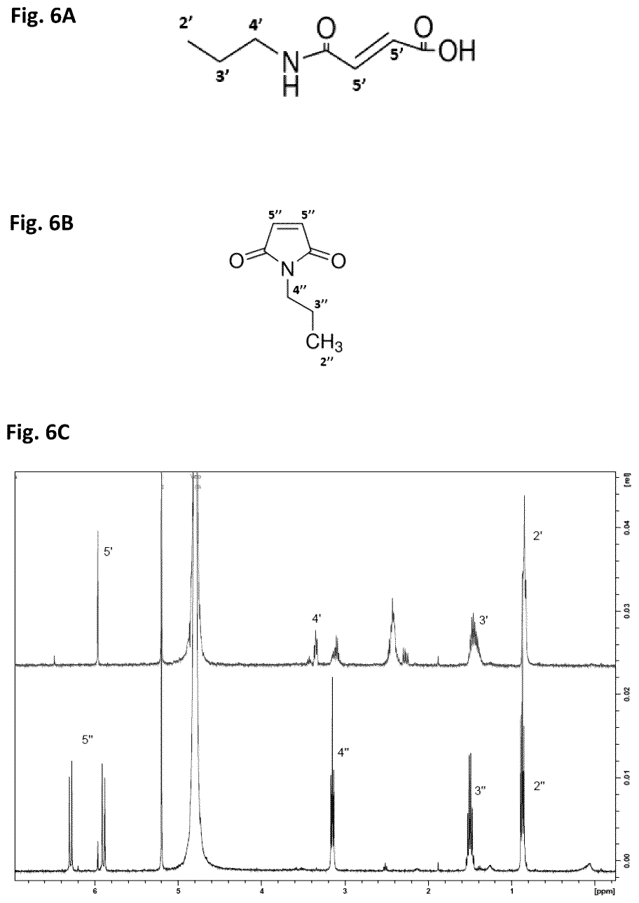

TABLE 13
H NMR signals and results of N-propylmaleimide in 0.4M NaOH
for two different samples.

| | Sample | | | |
|---|---|---|---|---|
| | 1 | | 2 | |
| Peak label in FIG. 6B | Trioxane | 5 | Trioxane | 5 |
| Peak location [ppm] | 5.2 | 5.9 and | 5.2 | 5.9 and |
| | | 6.3 | | 6.3 |
| Number of protons | 6 | 2 | 6 | 2 |
| Integral of NMR peak | 1.45 | 1.82 | 1.74 | 1.82 |
| mmol N-propylmaleimide from | — | 0.062 | — | 0.051 |
| NMR | | | | |

| | | |
|---|---|---|
| mmol N-propylmaleimide | 0.066 | 0.050 |
| introduced | | |
| % Error | 5.2 | 1.4 |

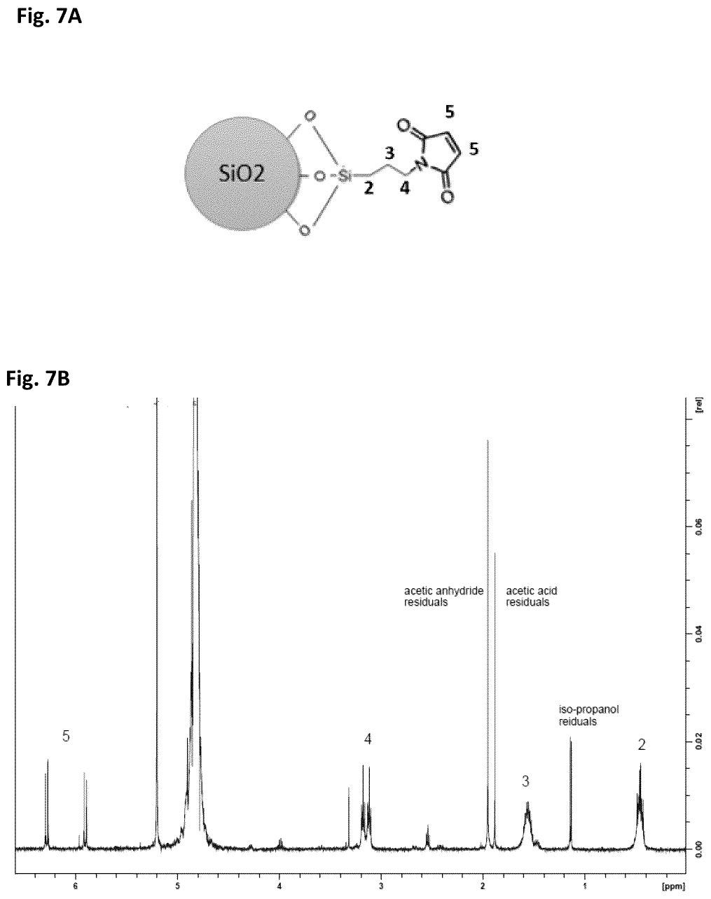

SiO₂-Maleimide after Dissolution in 0.4M NaOH:
Table 14 shows H-NMR signals and results of SiO₂-maleimide after treatment in 0.4M NaOH for three different samples. The trioxane integral value was the same for all the samples. The peak used to characterize and quantify the maleimide attachment was group labeled “5” as in FIG. 7B at 5.9 and 6.3 ppm. The integral value of group “5” is the sum of the two peaks, each representing one proton.

TABLE 14
H NMR signals and results of SiO₂-maleimide
after treatment in 0.4M NaOH

| Peak identification/ | | | | |
|---|---|---|---|---|
| sample number | Trioxane | Sample 1 | Sample 2 | Sample 3 |
| Number of protons | 6 | 2 | 2 | 2 |
| Integral of | 6 | 1.68 | 1.55 | 1.52 |
| NMR peak | | | | |
| mmol maleimide | — | 0.0060 | 0.0056 | 0.0054 |
| from NMR | | | | |
| # Maleimide | — | 0.85 | 0.79 | 0.79 |
| groups/nm² | | | | |

CysHex after Treatment in 0.4M NaOH:

Calibration curve in the range of 3.8-4.0 ppm (labelled group “a”):

$$I = 3.41 c - 0.08 \qquad ( D \text{.2} )$$

Calibration curve in the range of 4.1-4.4 ppm (labelled group “b”):

$$I = 4.81 c - 0.22 \qquad ( D \text{.3} )$$

Where I is the result of the integration of the NMR peak area, and c is the peptide concentration in terms of

$$\frac{mg CysHex}{ml NaOH solutin} .$$

TABLE 15
H NMR signals and results of SiO₂-CysHex after treatment in 0.4M NaOH.

| Peak identification in Fig. 8B | Trioxane | Group a | Group b |
|---|---|---|---|
| Peak/peak range location [ppm] | 5.2 | 3.8-4.0 | 4.1-4.4 |
| Number of protons | 6 | — | — |
| Integral of NMR peak | 4.62 | 3.10 | 4.27 |
| mg CysHex/ml NaOH solution (from cal. | — | 0.93** | 0.93 |
| curve) | | | |
| mmol CysHex | — | 0.00154*** | 0.00154 |

**Example of calculation of the mass of CysHex attached (mg/ml NaOH solution) after dissolution on the basis of the calibration curve for group “a”, eq. D.2:
$\frac{mg CysHex}{ml NaOH} = [ integral + 0.08 ] * \frac{1}{3.41} = [ 3.1 + 0.08 ] * \frac{1}{3.41} = 0.93$ ***Example of calculation of the number of mmol CysHex attached to the NP:
${n}_{CysHex} = \frac{mg CysHex}{ml NaOH} * \frac{1}{{Mw}_{CysHex}} * [ {V}_{NaOH} + {V}_{trioxane} ] ==$ $0.93 * \frac{1}{663.75} * [ 1 + 0.095 ] = 0.00154 mmol CysHex$ (D.4)

Where n_{CysHex} is number of mmols of CysHex obtained from the NMR, Mw_{CysHex} is the molar mass of CysHex [663.75 mg/mmol], V_{NaOH} and Vₜᵣᵢₒₓₐₙₑ are the volumes of 0.4M NaOH and trioxane solutions, 1 and 0.095 mL, respectively.

The average of mmol CysHex between the two peak groups is 0.00151. The mass of SiO₂-CysHex used was 9.67 mg. To convert this value to number of CysHex attached per nm², eq. 5.3 was used as follows:

$$\frac{\# CysHex}{{nm}^{2}} = \frac{{n}_{H NMR}}{{m}_{SiO 2 - CysHex}} * \frac{{N}_{A}}{{SA}_{silica}} = \frac{0.00 \; 154 m mol}{9 \text{.67} mg} * \frac{6.02 * 1 {0}^{20} \# groups / m mol}{2 * \frac{1 {0}^{17} {nm}^{2}}{mg}} = 0.48$$

### Silica Dissolution

To enable quantitative determination of the functional groups by means of liquid NMR, it is necessary to ascertain complete dissolution of the silica particle. Several NaOH concentrations and procedures were examined as follows: (A) 50 mg of silica NP were suspended in 5 mL 0.4M NaOH/H₂O and stirred vigorously for 3 hrs at 45° C. (B) 50 mg of silica NP were suspended in 5 mL 0.1M NaOH/H₂O and stirred vigorously for 3 hrs at 45° C. (C) 50 mg of silica NP were suspended in 5 mL 0.4M NaOH/H₂O and sonicated for 1 hr at room temperature.

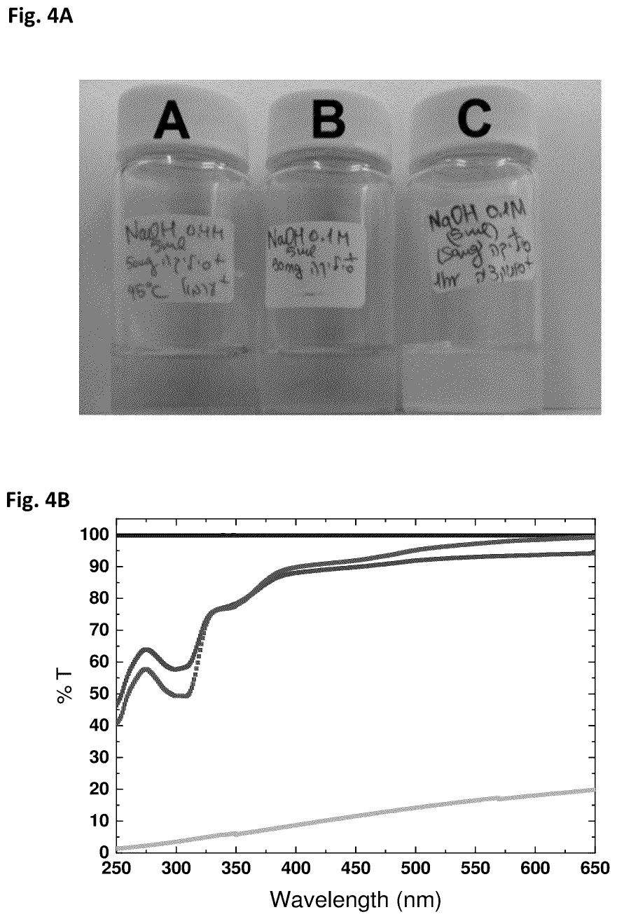

Samples A-C are shown in FIG. 4A. It is visible that the silica NPs did not dissolve completely in samples B and C. Sample B has a residue at the bottom of the vial, and sample C is cloudy, while sample A is visually clear to the naked eye. To confirm complete dissolution in sample A, UV-VIS transmission experiments were performed using Agilent Cary 5000 UV-VIS-NIR spectrophotometer (10 mm quartz cuvettes, 1 nm slit), and the results are shown in FIG. 4B. To confirm that the conditions used for sample A result in the optimal dissolution process, another sample was prepared employing somewhat harsher conditions: longer dissolution time of 18.5 hrs, and a higher NaOH concentration of 0.5M. A 0.4M NaOH solution in water was also examined as a control.

As shown in FIG. 4B, the NaOH solution (top blue line) does not absorb the radiated light at all and does not interfere with this examination's accuracy. The silica NP suspension in the water, shown in cyan, exhibits extremely low transmission (below 20%) throughout the entire wavelength range due to the size of the NPs aggregates, which prevents the radiated light from being transmitted. In the visible range above 400 nm the transmission of the silica solutions after dissolution in NaOH, shown in red and green, are above 90% in agreement with the visual inspection of sample A and confirms the dissolution of the NP. The lower transmission (˜50%) below 350 nm and the absorption peak around 300 nm are attributed to absorption by the sodium silicate (Na₂SO₃) molecules, products of the silica reaction with NaOH. The improvement in transmission as result of six-fold longer exposure time and 25% stronger base is negligible as clearly demonstrated in FIG. 4B. Thus, it was decided to carry out dissolution experiments under stirring conditions for 3 hrs using 0.4M NaOH solution.

### APTMS

Three types of ¹H NMR experiments were carried out and the corresponding spectra obtained: 1) the spectrum of APTMS molecule dissolved in D₂O (FIG. 5D top curve green); 2) the spectrum of APTMS molecule after being exposed to the dissolution process of a bare silica NP in 0.4M NaOH/D₂O (FIG. 5D middle curve, red); 3) the spectrum following the dissolution of SiO₂—NH₂ in 0.4M NaOH/D₂O (FIG. 5D bottom curve, blue). The first experiment served to identify and assign the relevant peaks of the APTMS molecule. The second experiment examined the effect of the dissolution process on the peaks identified by the first experiment. Both experiments also served to determine the accuracy of the NMR method by comparing the amount of amine groups determined from the NMR spectra with the known amounts of APTMS molecules introduced. The third experiment allowed the actual determination of the amount of amine groups successfully attached to the silica surface.

Dioxane (FIG. 5B) was used as the external standard in the first two experiments (protons labeled “#” in FIG. 5B, and peak in 5D, resonance at 3.7 ppm). Trioxane (FIG. 5C) was used as the external standard in the third experiment (protons labeled “*” in FIG. 5C and peak in FIG. 5D, resonance at 5.2 ppm). Residual H₂O resonance is detected at 4.8 ppm. The different protons on APTMS are labeled for each peak in the spectra and identified in FIG. 5A. The peak corresponding to methylene group adjacent to the terminal amine (proton 4) is the only one affected by the dissolution process and shifts from 2.5 ppm in D₂O to 2.8 ppm following the NaOH dissolution procedure. This shift is attributed to the protonation processes undergone by its neighboring primary amine group when shifting from slightly acidic to strongly basic environment. The two additional peaks “2” at 0.4 ppm and “3” at 1.5 ppm corresponding to the remaining methylene groups of APTMS, are unaffected by the change of solvent or the dissolution process. No difference is observed in the protons of all three methylene groups whether attached or detached from the silica NP (experiments 2 and 3). The peak at 3.2 ppm, labeled “1”, is assigned to the methoxy groups of APTMS molecules. In the bottom (blue) spectrum corresponding to dissolved SiO₂—NH₂ this peak has practically disappeared indicating the absence of methoxy groups. As shown above, by both XPS and TGA, on average only two out of the three methoxy groups of APTMS have reacted with the silanols. A possible explanation for the disappearance of the remaining third methoxy group is its consumption by base catalyzed hydrolysis reactions. Alternatively, the basic environment and elevated temperature may have facilitated its reaction with surface silanols prior to the particle dissolution. Yet, none of these impact the ability to determine the surface amine concentration.

Accuracy of the ¹H qNMR Method in Determination of APTMS Silica Surface Density

The accuracy of the H qNMR method was tested by comparing the value of known amounts of the APTMS molecules dissolved in D₂O in experiments (1) and (2) to the values obtained from the integration values in the corresponding NMR spectra. The number of APTMS molecules in the sample was obtained from the average of the values calculated for each one of the four peaks separately using eq. 5.2.

The uncertainty in using ¹H qNMR of APTMS molecules was estimated as 1.5% and 3.0%, for experiments (1) and (2) respectively. The integration values and detailed calculations used to obtain these estimates are presented herein. Compared to the uncertainty in using TGA to quantify APTMS, which is 20%, the H qNMR has superior accuracy.

Amine Surface Density of SiO₂—NH₂

Once the accuracy of the NMR technique has been established the unknown amount of amine groups on the silica NP surface was determined. The quantification of amine attachment to the silica surface was obtained from the average of the values computed for the three characteristic resonances at 0.4, 1.4, and 2.5 ppm with trioxane as the internal standard (peak at 5.2 ppm in bottom spectrum in FIG. 5D). From eq. 5.2 and eq. 5.3 below a value of 2.9 amine groups per nm² was determined:

$$\frac{\# functional groups}{{nm}^{2}} = \frac{{n}_{H NMR}}{{m}_{SiO 2 - functional group}} * \frac{{N}_{A}}{{SA}_{silica}} \qquad Eq . 5.3$$

Here n_{H NMR} is the number of mols of APTMS calculated from the H NMR results, m_{SiO2-functional group} is the mass of functionalized silica in the sample, SA_{silica} the specific surface area of silica NP (2*10²⁰ nm²/gr), and N_{A} the Avogadro number. The data used to obtain the number of amine groups attached per nm² is presented above.

The value of 2.9 amine groups/nm² obtained by H qNMR is 5% higher than the upper limit obtained by TGA and indicates a somewhat higher efficiency of amine functionalization of the silica NP than determined by the TGA method.

### Maleimide

The synthesis of SiO₂-maleimide was achieved in two steps, functionalization with a maleamic acid group followed by closing of the maleimide ring. To identify the relevant NMR peaks the reagents N-propylmaleamic acid (FIG. 6A) and N-propylmaleimide (FIG. 6B) were used. The former has similar structures to the three-carbon backbone of APTMS attached to a maleamic acid group after reaction with an amine end group and the latter to a closed maleimide ring

To find unique peaks associated with the maleamic acid and the maleimide groups after treatment with 0.4M NaOH/D₂O and not overlapping with the APTMS signals, N-propylmaleamic acid and N-propylmaleimide spectra were compared, as shown in FIG. 6C. Signals at resonances of approximately 0.9, 1.5, and 3.1 ppm and labeled 2′, 3′, 4′ or 2″, 3″, 4″, in FIGS. 6A-C are overlapping for both reagents. Two large peaks at approximately 2.4 and 3.0 ppm are observed only in the maleamic acid spectra. The singlet labeled 5′ at 5.95 is assigned to the double bond in the maleamic acid. The triplet at 5.9 and doublet at 6.3 ppm, labeled 5″, observed only for N-propylmaleimide, are assigned to the protons of the maleimide group. These peaks also do not overlap with any of the APTMS peaks as can be realized by comparison to FIG. 5D. These three latter peaks were used to verify and quantify the attachment of the maleamic acid to the silica NP and its conversion into a maleimide at the second functionalization step.

Accuracy of the ¹H qNMR Method in Quantitative Determination of Maleimide Concentration:

The accuracy of quantitative determination of the number of maleimide groups following NaOH treatment was determined by comparison of known amounts of N-propylmaleimide molecules to the number obtained from spectra integration as described above. The average mmol of N-propylmaleimide was obtained from the integration of the two characteristic resonances at 5.9 and 6.3 ppm compared to that of the external standard (trioxane) at 5.2 ppm. The error based on the average of two different samples was estimated as 3.3%. The data used to obtain this value can be found above (Table 13). As also noted above, the error in quantifying maleimide groups by H qNMR is significantly lower than that of TGA which is estimated as 20%.

### Maleimide Surface Density of SiO₂-Maleimide

The functionalization of silica surface with maleimide groups are quantified by the two protons of the double bond in the maleimide group labeled “5” at 5.90 and 6.30 ppm in FIG. 6A. The NMR full NMR spectrum is depicted in FIG. 7B. Peaks corresponding to residual solvents from the synthesis are identified. The peaks at 4.8 and 5.2 ppm correspond to residual water and trioxane respectively. The small unidentified peak at 3.4 ppm can possibly belong to residual maleamic acid.

Based on the average of three different samples the surface density of maleimide groups on the silica was determined as 0.81±0.03 maleimide groups/nm². The data used to obtain this value can be found above (Table 14).

The value obtained by the NMR is 30% lower than the lower limit obtained by the TGA. The difference may be due to solvents and contaminants detected by the NMR as shown in FIG. 7B. Peaks at 1.10, 1.85, and 1.90 were identified as iso-propanol, acetic acid, and acetic anhydride respectively, solvent residuals from the different synthesis steps. Two additional unidentified peaks were found at 2.50, and 3.30 ppm. These impurities can bias the TGA results upwards by attributing all mass loss to the maleimide functionalization. At the same time, it may bias the NMR results downwards since the actual mass of functionalized silica is smaller than the value of m_{SiO2-maleimide} used in eq. 5.3.

### CysHex

Accuracy of Quantitative Determination of CysHex Surface Density by H qNMR

The same set of three experiments used in previous analyses has been employed here as well. The top spectrum in FIG. 8A (green) corresponds to the CysHex peptide dissolved in D₂O. Due to the large number of different protons in this molecule and the multitude of resulting peaks those were not assigned. Instead, significant peaks which do not overlap with any of the peaks in the spectra of the previous functionalization steps were identified. In the spectra in FIG. 5D and FIG. 7B, no significant peaks are detected between 3.5 ppm and 4.67 ppm. Whereas here, two groups of peaks are observed between 3.8-4.0 ppm and 4.1-4.4 ppm. These peaks are best suited for the analysis of the CysHex functionalization.

The next step involved subjecting the peptide to the dissolution process in NaOH solution (experiment 2, FIG. 8A bottom spectrum—red). In contrast to the case of the amine and maleimide functionalization, the dissolution process had a major impact on the different peaks of the peptide as evident from comparison between the two spectra in FIG. 8A. The different peaks shifted, some of them merged and overlapped, their relative sizes changed, and their shapes were modified. The NMR spectrum of the peptide functionalized silica particle after silica dissolution is depicted in FIG. 8B showing the same smeared, overlapping characteristic peaks between 3.8-4.0 ppm (named group “a”) and 4.1-4.4 ppm (group “b”) marked in the figure.

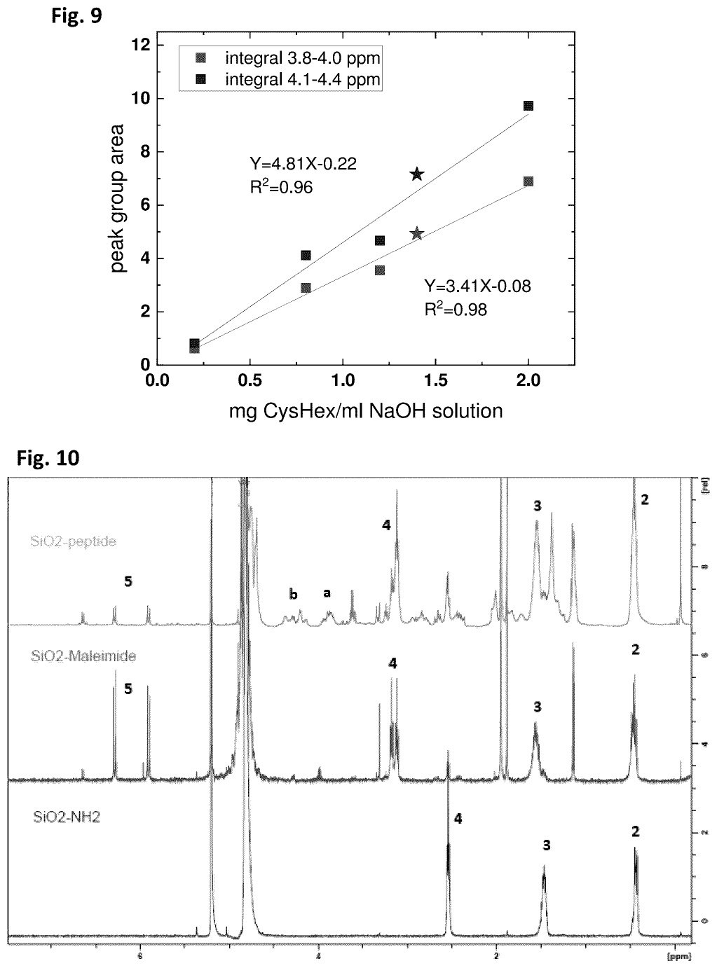

The distortion and smearing of the peaks made it difficult to quantify the amount of peptide using eq. 5.2. Due to this obstacle, generation of a calibration curve by integrating over the entire group of peaks a or b using different known concentrations of peptide in NaOH solutions and using trioxane as the external standard were used. Calibration curves are presented in FIG. 9 and the data used to generate these curves are provided below. These two calibration curves were subsequently used jointly to determine the amount of peptide attached to the silica NP. To estimate the error involved in using the calibration curves the concentration of peptide in a test sample obtained using the calibration curves was compared to the known value of 1.4 mg CysHex/mL NaOH solution. An overestimate of 5.0% and 9.3% was obtained by using the calibrations for groups a and b, respectively (cf. below). The justification for the selection of these two peak groups is further demonstrated in FIG. 10 showing the lack of overlap with the other functional groups attached in the previous synthesis steps.

### Accuracy of CysHex Determination by the Calibration Curves for Peptide in NaOH Solution

A sample with concentration of 1.40 mg CysHex/ml NaOH solution was used to check the accuracy of the calibration curves described by equations D.2 and D.3.

TABLE 16
H NMR signals and results for a sample with concentration of 1.40 mg
CysHex/ml NaOH solution:

| Peak group | Group a | Group b |
|---|---|---|
| Peak/peak range [ppm] | 3.8-4.0 | 4.1-4.4 |
| I_{NMR}, NMR integral | 4.92 | 7.16 |
| Peptide concentration estimated by the calibration | 1.47* | 1.53* |
| curve | | |
| % error in concentration obtained by the | −5.00% | −9.29% |
| calibration curves | | |

Calculation of the peptide concentration from calibration curve D.2:

$$\begin{matrix}Group a : c = ( 4.92 + 0.08 ) / 3.42 = 1.47 \\ Group b : c = ( 7.16 + 0.22 ) / 4.81 = 1.53\end{matrix}$$

### CysHex Surface Density of SiO₂-CysHex

The integration values of the areas under the peaks of groups a and b in FIG. 8B yielded values of 3.10 and 4.27 respectively. Using the corresponding calibration curves both yielded the same value for the peptide concentration of 0.93 mg CysHex/mL solution. The latter value is equivalent to 0.48±0.03 peptide groups/nm². The detailed calculation is provided above. This value is in general agreement with the values obtained by TGA (0.42-0.36 peptide groups/nm²).

### The Progression of the Synthesis as Evaluated by ¹H NMR

The ¹H NMR spectra obtained for SiO₂—NH₂, SiO₂-maleimide and SiO₂-CysHex compared in FIG. 10, allow the identification of the progression of the synthesis from one stage to the other. The labeled peak assignments can be found in FIGS. 5A, 7A and 8B.

The outcome of the first synthesis step is the functionalization of the silica by APTMS. It results in three peaks labelled 2, 3, and 4 in the bottom spectrum corresponding to the three methylene groups. The first two are also observed in the same position in the two other spectra. The next functionalization step resulted in adding a maleimide group to the tethered chain. As result methylene group 4 which is the closest to the amine group shifted from 2.5 ppm to 3.2 ppm and is observed in both in the top SiO₂-CysHex spectra as well. Peaks 5 corresponding to the maleimide ring are also visible in the top spectrum. This is a clear indication that not all maleimide groups have been consumed by the reaction with the cysteine terminus of the peptide.

### Surface Coverage

Theoretically, the number of peptides bound to the silica surface should be similar to the number of maleimide groups since each maleimide group may react with one peptide molecule.

Yet, the surface density of maleimides is 0.81±0.03 groups/nm² and that of peptide is 0.48±0.03 groups/nm². Thus, only 60% of the maleimides have reacted as already realized by the presence of the relevant peaks in the SiO₂-CysHex NMR spectrum. It is of importance to determine whether the number of peptides attached to the surface has been limited as result of the reaction kinetics or due to steric effects. For this reason, based on steric considerations the maximum number of tethered peptide chains per particle was determined.

A diffusion coefficient value of 3*10⁻¹⁰ m²/sec was obtained for the CysHex peptide molecule by means of Diffusion Ordered Spectroscopy (DOSY) NMR. Application of the Stokes-Einstein equation under the assumption of a spherical excluded volume yields an estimate of Rₚ=0.8 nm for the dimensions of the peptide molecule. Hence, the area of the peptide projected on the NP surface is Aₚ=πRₚ²≈2 nm². The surface area of a single silica NP with a radius of 6 nm is 450 nm². As result the number of close-packed peptide chains per particle is approximately 225 peptide molecules per NP.

The H NMR surface density analysis discussed above yielded 0.48 molecules per nm² or 0.48*450≈215 peptide molecules per NP. These results support the notion that the surface coverage of peptide molecules successfully attached to the silica NP by the procedure described herein is close to the maximum loading and additional loading is hindered by steric considerations.

### Determination of Surface Density by Elemental Analysis

Elemental Analysis (EA) CHNS is considered a high accuracy analytical technique capable of determining the mass fractions of Carbon, Hydrogen, Nitrogen and Sulphur in organic samples. In view of the difficulties encountered in the direct analysis of the H NMR spectrum of SiO₂-CysHex following dissolution, it was expected EA could be useful for quantitative determination of the functionalization of the silica with the CysHex peptide by determining the amount of sulfur addition to the NP. Sulfur being found only in the thiol side group of cysteine.

To test the accuracy of this technique, the unattached peptide molecule was analyzed, and the values compared to the known mass fractions of the atoms composing the peptide. As shown in Table 17 the error for this relatively simple material was considerably higher than that obtained from the NMR technique aided by the calibration curves. As result it was opted not to employ EA.

TABLE 17
comparison of known mass fraction of N, C, H, S in CysHex
peptide and values determined by elemental analysis.

| | % wt in peptide | | |
|---|---|---|---|
| | from chemical | % wt from | |
| | structure | EA | Error (%) |
| N | 18.6 | 14.8 | 20 |
| C | 44.2 | 38.6 | 13 |
| H | 6.6 | 6.1 | 8 |
| S | 4.7 | 4.4 | 6 |

In Table 18, the values obtained by the ¹H qNMR are compared to the estimates obtained from TGA measurements. Based on the extensive evaluations of the technique it is assumed that the values obtained by the ¹H qNMR represent accurately the particle surface coverage.

TABLE 18
NP surface coverage after each synthesis step quantified
using TGA and 1H qNMR.

| | TGA | | |
|---|---|---|---|
| | (# groups/nm²) | | H NMR |
| | Upper limit | Lower limit | (# groups/nm²) |
| SiO₂—NH₂ | 2.77 | 2.18 | 2.90 |
| SiO₂-maleimide | 1.51 | 1.19 | 0.81 |
| SiO₂-CysHex | 0.42 | 0.36 | 0.48 |

### Example 3—Phosphate Capture Capability of the Particles

The ability of the peptide to bind phosphate in GI fluid simulants was tested for both the free unbound peptide, and the peptide tethered to the silica NP. The former by ³¹P NMR technique and the latter by colorimetric methods. Ion chromatography methods are also disclosed in conjunction with the latter.

### Free Peptide-³¹P NMR

The capability of a free unbound peptide to capture phosphate in the intestinal simulant solutions was examined using P NMR. These experiments aimed to investigate the effect of the background solution containing many components on the ability of the peptide to bind phosphate. As discussed above, binding is qualitatively evaluated by the decrease in T₂ relaxation, or the corresponding widening of the peak associated with the phosphorus nuclei in the molecule and calculated as follows:

$$peak width ratio = \frac{{PW}_{phosphurus + peptide solution}}{{PW}_{phosphurus only solution}} \qquad Eq . 6.1$$

here PW is the peak width at half height of the phosphorus containing compound (phosphate or phospholipid) normalized by the peak width of the external standard (phosphoric acid).

### pH Effect on Phosphate Chemical Shift

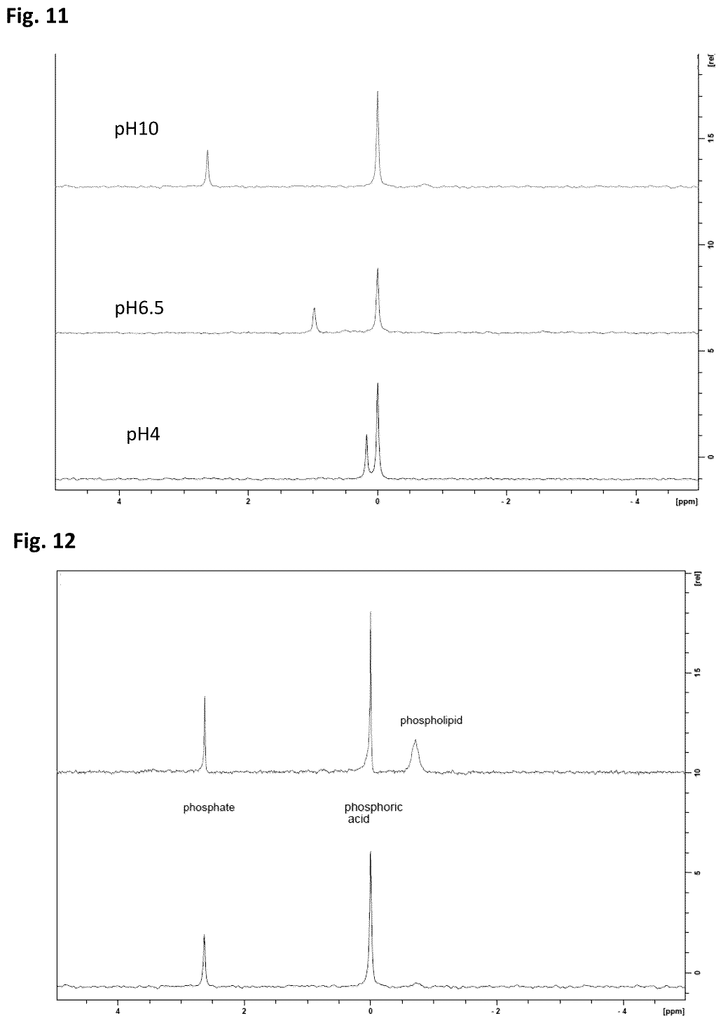

Samples were prepared in a range of pH values to examine performance of the peptide at different surroundings. The chemical shift of the phosphate peak depends on the ionization state of the molecule. As the pH decreases, the chemical shift of the phosphate peak decreases, as shown in FIG. 11 for a 2.6 mM Na₂HPO₄ phosphate (the same phosphate was used in all experiments) in FAS solution. The chemical shift of the phosphate peak decreases from 2.6 ppm at pH 10 (top spectrum-green) to 0.2 ppm at pH 4 (bottom-blue). At pH values lower than 4, the peaks of the phosphate and the phosphoric acid external standard overlap, preventing accurate determination of peak widths. Therefore, the lowest examined pH was 4.

### Peak Characterization in Intestinal Fluid Simulants

FIG. 12 depicts an example of ³¹P NMR spectra of 2.6 mM phosphate in FAS or FES solutions at pH 10. In both spectra, the phosphate peak is observed at 2.6 ppm. In addition, a significant peak is observed at −0.7 ppm in the FES solutions, assigned to the phospholipids. Due to the tenfold smaller phospholipid concentration in the FAS solution, FES: 2 mM and FAS:0.2 mM) the phospholipid peak for the latter is hardly noticeable. In view of the presence of significant amounts of phospholipid in the GI simulants, investigation of their interaction with the phosphate binding peptide was performed.

### Phospholipid Influence on Phosphate Binding in FES Solutions

FIG. 13 depicts the widening of the phospholipid signal in FES solutions as a function of pH in the presence of only peptide (squares) or peptide and phosphate (triangles). Phospholipid concentration was kept at 2 mM, peptide and phosphate concentrations were 13.1 mM and 2.6 mM respectively. The widening of the phospholipid peak in the absence of phosphate, indicates that the peptide binds the phospholipid quite effectively over the entire relevant pH range at a molecular ratio of 6.5 peptide molecules per phospholipid molecule. The lowest binding is observed at pH ˜6 which is the relevant pH in the intestine. When phosphate is also present (FIG. 13, triangles) there is a reduction in phospholipid binding apart from pH=12 (which is of little importance from a physiological perspective), probably as result of competition between the phospholipid and phosphate over the peptide binding. Since the molar amounts of phospholipids and phosphate are close, it was concluded that the efficiency of phosphate binding by the peptide is higher than that of the phospholipid and despite the large excess of peptide molecules, phospholipid minding is hindered by the presence of the phosphate.

Due to the relevance of the behavior at pH≈6, this data point in FIG. 13 represents the average of three independent measurements while the rest of the data in the figure are based on single measurements. The value obtained for the peak width of the phospholipid in the presence of peptide and phosphate at pH=6 is 0.93±0.15 indicating that phosphate binding has practically eliminated binding of the phospholipid in the most relevant pH value.

### Phosphate Binding in Intestinal Fluid Simulants

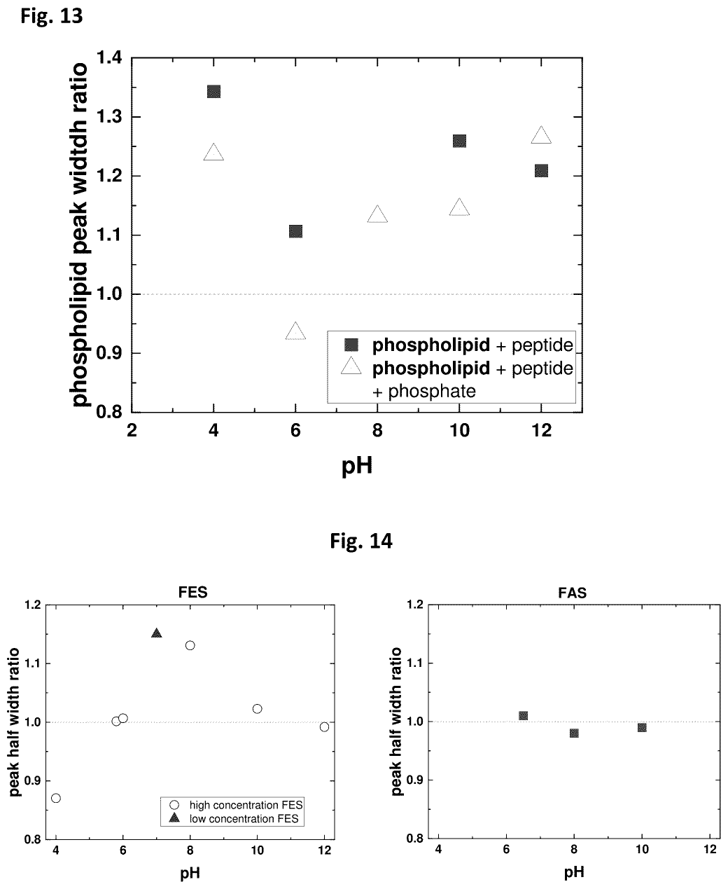

The widening of the phosphate peak as indication of its capture by the peptide was tested. The phosphate peak widening in FES solution depicted in the left-hand side of FIG. 14 shows that the widening is pH dependent and is maximal at pH values commensurate with values in the GI tract. However, the phosphate peak in FAS solutions shows no widening as observed on the right-hand side of FIG. 14. These results seem to indicate little or no binding of phosphate to the peptide in the FAS solution.

One possible explanation to the lack of binding relates to the extremely high concentration of NaCl in the GI fluid simulants solutions, which may interfere with phosphate binding by screening out electrostatic interactions. In order to test this hypothesis, NaCl was diluted by a factor of 20 as described above. This dilution had little impact on phosphate binding in either FAS or FES solutions. Except for the case of pH=7 in FES solution, indicated by the triangle in FIG. 14, the rest of the data shows no widening of the phosphate peak. Furthermore, in some cases the phosphate peak width became narrower, an indication of a larger rather than smaller T₂ relaxation of the phosphate molecule.

Unlike in pure water, the composition of FAS or FES solutions prevent the peptide from capturing phosphate except for a very limited range of pH values around 7-8 and only in FES. It may be that the NMR techniques employed here is unable to determine phosphate binding to the peptide.

### SiO₂-CysHex-Colorimetric

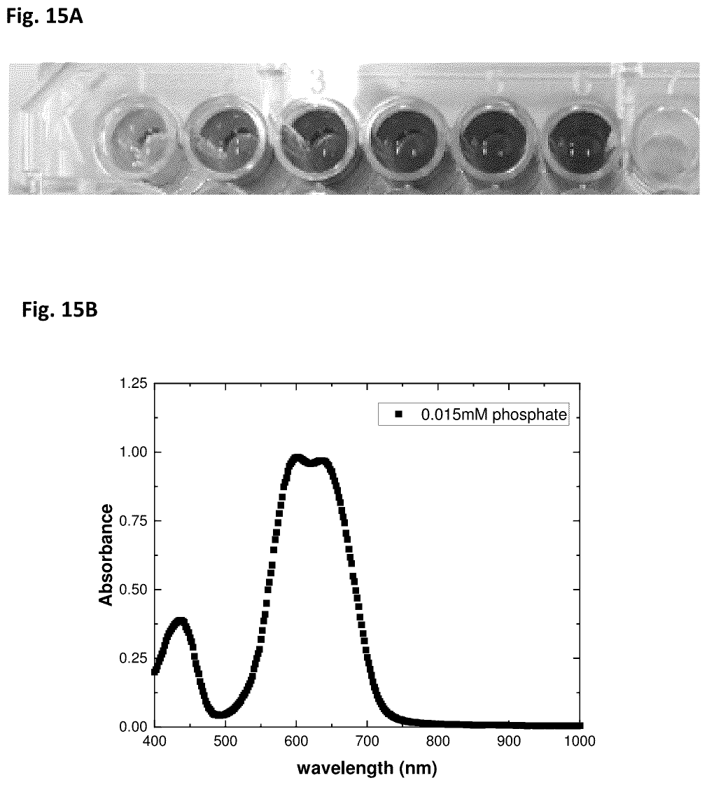

The phosphate binding efficiency of SiO₂-CysHex in intestinal and digestive fluid simulants was examined by a colorimetric reaction using molybdenum blue and quantified by absorbance spectroscopy. In FIG. 15A, samples for the calibration curve after reaction with the reagent are shown. A gradient of blue shades can be seen, with the highest phosphate concentration on the right.

FIG. 15B is an example of the absorbency spectrum of a solution containing 0.015 mM phosphate, mixed with reagents to produce a molybdenum blue complex. This complex shows two unique peaks with a maximal height at 420 and 650 nm. For the analysis here, the latter peak was chosen. For each set of measurements, a calibration curve between absorbance intensity and phosphate concentration was generated as demonstrated below (Table 19). The percent of phosphate captured was determined from the difference between the initial phosphate concentration in the solution and the concentration remaining in solution at the end of the binding experiment as detailed below:

Table 19 shows Phosphate binding obtained by the absorbance of the molybdenum blue complex for three repetitions in FAS solution at pH=6. C₀-Initial phosphate solution, C_{f}-solution at the end of binding experiment.

TABLE 19

| | Sample 1 | | Sample 2 | | Sample 3 | |
|---|---|---|---|---|---|---|
| | C₀ | C_{f} | C₀ | C_{f} | C₀ | C_{f} |
| Absorbance at | 0.900 | 0.232 | 0.859 | 0.432 | 0.757 | 0.316 |
| 650 nm | | | | | | |
| C [mM] | 0.0144 | 0.0027* | 0.0137 | 0.0062 | 0.0119 | 0.0041 |
| % Phosphate | — | 81.4 | — | 54.7 | — | 65.1 |
| captured | | | | | | |

*Phosphate concentration conversion from absorbance reading by using a calibration curve:

$$C = \frac{[ 0.232 - 0.079 ]}{57.006} = 0.0027$$

The average value is 67.1%±11.0% phosphate captured.

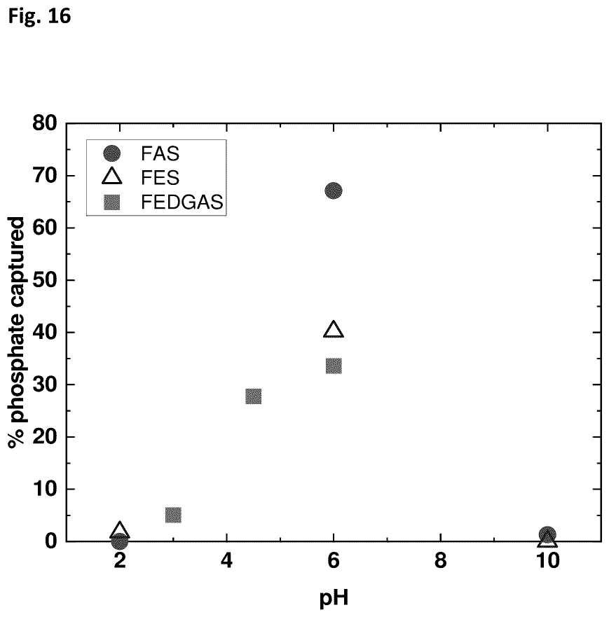

The amount of phosphate binding was determined for the three GI tract fluid simulants as function of pH. The results are depicted in FIG. 16. In all experiments the molar ratio between peptide and phosphate was kept at 5:1 and the phosphate concentration were 0.15 mM. In agreement with the NMR experiments practically no phosphate binding is observed for all three simulants at pH below 4 or above 10. Yet in contrast to the NMR results very high binding efficiency is demonstrated in the FAS solution at pH=6. Substantial phosphate binding is observed in all three simulants at pH 6 which is the relevant pH of the intestine.

Phosphate binding in FES solution is less effective than in FAS, probably due to the competition with phospholipids. For FEDGAS solution, substantial binding is obtained at pH 4.5 and 6 which correspond to 75% or 100% full stomach, respectively.

A quantitative measure of the ability of the peptide functionalized silica to bind phosphate is the mass of phosphate captured per mass of SiO₂-CysHex as computed by eq. 5.5:

$$\frac{mg phosphate captured}{gr {SiO}_{2} - CysHex} = \frac{\% phosphate captured}{100} * {C}_{0} * {Mw}_{ph} * \frac{V}{{m}_{{SiO}_{2} - CysHex}} \qquad ( 5.5 )$$

Where C₀ is the initial phosphate concentration, Mwₚₕ is the molar mass of the phosphate, V is the solution volume, and m_{SiO2-CysHex} is the mass of the SiO₂-CysHex NP.

A calculation example of the mass of phosphate (in mg) captured by 1 gr of the peptide functionalized silica NP using eq. 5.5 in FAS solution at pH=6:

$$\begin{matrix}\frac{mg phosphate captured}{gr {SiO}_{2} - CysHex} = \frac{\% phosphate captured}{100} * {C}_{0} * {Mw}_{phosphate} * \frac{{V}_{solution}}{{m}_{{SiO}_{2} - CysHex}} = \\ = \frac{67.1 \%}{100} * 0.15 mM * 95 \frac{mg}{m mol} * \frac{1 * {10}^{- 3} L}{3.69 * {10}^{- 3} gr} = 2.59\end{matrix}$$

The values obtained at pH=6 in the three different GI tract simulants are presented in Table 20. The value in FAS solution is an average of three different samples, FES and FEDGAS solutions are average of two different samples. The variation between the values of the duplicate measurements was approximately 5%.

TABLE 20
Average amount of phosphate captured in the
fluid simulants at pH = 6.

| | FAS | FES | FEDGAS |
|---|---|---|---|
| mg phosphate/gr | 2.6 | 1.5 | 1.3 |
| SiO₂-CysHex | | | |

Based on these results it may be argued that on the one hand, it is preferable to take the PhB after food consumption due to higher efficiency of phosphate binding in the stomach when it is full (at pH 6). On the other hand, the phosphate binding is lower in FES solution corresponding to the GI environment after a meal in comparison to FAS solution which simulates the state of the GI before a meal.

### Example 4—Comparison with Clinically Used PhB-Lanthanum

The phosphate-binding efficiency of the SiO₂-CysHex system, was compared to that of lanthanum carbonate. The results are presented in Table 21. Values are an average of two independent measures with 5% variation. Tests were carried out at pH=6 or pH=7.5, both in the range of values corresponding to the conditions in the intestine. The results are presented in Table 21, which shows a comparison between phosphate binding capacity of different phosphate binders in intestinal fluid simulants. Values are an average of two measurements (±2.5%).

TABLE 21

| | Phosphate binding effectiveness | |
|---|---|---|
| | [mg phosphate/gr PhB] | |
| | FAS | FES |
| Lanthanum | 4.1 | 0.9 |
| SiO₂-CysHex | 2.6 | 1.5 |

The results clearly show that the phosphate binding efficiency of the SiO₂-CysHex particles is in the same order of magnitude as lanthanum carbonate and it is twice as effective in the fed state.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1.-31. (canceled)

32. A composition comprising silica nanoparticles conjugated to a phosphate binding hexapeptide (PBH) via a linker amino acid residue.

33. The composition according to claim 32, wherein the linker amino acid residue comprises a Cysteine amino acid.

34. The composition according to claim 32, wherein the hexapeptide comprises an amino acid sequence as denoted by SEQ ID NO: 1.

35. The composition according to claim 32, wherein the hexapeptide comprises a Cys linker amino acid residue at the N-terminal thereof, said conjugated peptide comprises an amino acid sequence as denoted by SEQ ID NO: 2.

36. The composition according to claim 32, wherein the silica nanoparticles are in an average size of about 5-200 nm.

37. The composition according to claim 36, wherein the silica nanoparticles have an average size of about 7-15 nm.

38. The composition according to claim 32, wherein the silica nanoparticles have an average surface area in the range of about 100-400 m²/gr.

39. The composition according to claim 32, wherein each nanoparticle is conjugated to at least about 200 PBH molecules.

40. A method of treating hyperphosphatemia, the method comprising administering a pharmaceutically effective amount of the composition according to claim 32 to a subject in need thereof.

41. The method according to claim 40, wherein the administration is systemic.

42. The method according to claim 40, wherein the administration is enteral.

43. The method according to claim 40, wherein the administration is by oral administration.

44. The method according to claim 40, wherein the subject is a subject afflicted with chronic kidney disease (CKD),

45. A process for the preparation of silica nanoparticles conjugated to a phosphate binding hexapeptide (PBH), the process comprises the steps of:
substituting hydroxyl group(s) on the silica nanoparticles surface with amine group(s);
reacting maleic anhydride with the amine group(s) of the silica nanoparticles surface to yield maleimide end group(s); and
attaching an hexapeptide having a cysteine amino acid end group linker to the maleimide functionalized nanoparticles, via said cysteine amino acid end group linker, to thereby form Silica-CysHex particles.

46. The process according to claim 45, wherein the hexapeptide comprises an amino acid sequence as denoted by SEQ ID NO: 1.

47. The process according to claim 45, wherein the silica nanoparticles have an average size of about 5-200 nm.

48. The process according to claim 45, wherein the silica nanoparticles have an average surface area in the range of about 100-400 m²/gr.

49. The process according to claim 45, wherein each nanoparticle is conjugated to at least about 200 hexapeptide molecules.

50. The process according to claim 45, further comprising a step of drying the resulting Silica-CysHex particles.

51. The process according to claim 50, wherein the drying comprises lyophilization of the Silica-CysHex particles.
